(19) United States
(12) Patent Application Publication
(10) Pub. No.: US 2025/0297303 A1
(43) Pub. Date: Sep. 25, 2025

(54) COMPOSITIONS AND METHODS FOR SELECTIVE EXTRACTION OF OLIGONUCLEOTIDES FROM COMPLEX MATRICES

(71) Applicant: CEPHEID, Sunnyvale, CA (US)

(72) Inventors: Andrea CUPPOLETTI, Sunnyvale, CA (US); Lucian ORBAI, Sunnyvale, CA (US); Lei PEI, Sunnyvale, CA (US); Alex I. KUTYAVIN, Sunnyvale, CA (US); Alex HIRSCHI, Redwood City, CA (US); Isita JHULKI BALLARD, Belmont, CA (US)

(21) Appl. No.: 19/086,844

(22) Filed: Mar. 21, 2025

Related U.S. Application Data
(60) Provisional application No. 63/568,277, filed on Mar. 21, 2024.

(51) Int. Cl. C12Q 1/6844; C12Q 1/6806; C12Q 1/6876

(52) U.S. Cl. CPC C12Q 1/6844; C12Q 1/6806; C12Q 1/6876

(57) ABSTRACT

Compositions and methods for isolating and detecting nucleic acid in a biological sample are provided. The compositions and methods utilize a solid support and an affinity reagent for isolating and detecting the nucleic acid.

### CROSS REFERENCE TO RELATED APPLICATIONS

This application is being filed on Mar. 21, 2025, as a U.S. Nonprovisional application and claims the benefit of U.S. Provisional Application No. 63/568,277, filed on Mar. 21, 2024, the disclosure of which is hereby incorporated by reference in its entirety. To the extent appropriate, a claim of priority is made to the above-disclosed application.

### FIELD OF THE DISCLOSURE

The invention relates generally to the field of molecular biology. In certain embodiments the invention provides devices, kits, and methods relating to the isolation and detection of nucleic acids.

### BACKGROUND

Some existing nucleic acid sample preparation methods involve the use of an automated or semi-automated cartridge for handling and movement of the sample and reagents used. In general, after a biological sample is lysed, nucleic acids are bound to a filter material in the cartridge, optionally washed, and eluted for detection. Frequently, the buffers used in the cartridge contain substances such as PEG, guanidine, or other chaotropes which carry over into the final detection reaction, affecting its efficacy even when the substances are present at low concentrations. It is desirable to avoid the co-elution of such substances into the eluate, and it would be especially desirable to eliminate the need for the use of such substances in the cartridge in the first place.

Additionally, it would be desirable to have enhanced control over the properties (e.g. density or binding capacity) of the filter material used in isolation of nucleic acids. To date, control over the surface properties of a material is generally achieved by altering the chemistry of the solution-phase or gas-phase methods used to prepare such material. These methods, however, can be cumbersome and expensive to implement at manufacturing scale, and the extent of the customization allowed is limited by increases in manufacturing complexity. Certain embodiments of the invention described herein provide for these and other needs. Other embodiments of the invention described herein provide for devices and kits which may be used for isolating nucleic acids from a sample. Still other embodiments of the invention provide for the detection of a nucleic acid in a sample.

### SUMMARY

Described herein are compositions, methods, and devices for isolating and purifying nucleic acid from a sample. The compositions, methods, and devices utilize a solid support that is optionally modified, an affinity reagent comprising a solid support binding moiety and a DNA binding moiety. Additionally, methods for modifying and controlling properties of the solid support material for nucleic acid extraction and isolation are provided. The methods utilize changes to the assay and solid support chemistry, allowing for much greater flexibility during assay development, and providing great cost-savings in manufacturing, as a single solid support could be used for many different assays.

In some aspects, methods of isolating nucleic acid from a sample comprising: contacting a solid support with an affinity reagent and the sample, wherein the affinity reagent comprises a first moiety that interacts with the solid support and a second moiety that interacts with nucleic acid in the sample, and concentrating the nucleic acid onto the solid support are disclosed. The affinity reagent can further comprise a linker that interacts with a solvent and increases solubility of the affinity reagent. The solid support can comprise a functional surface group that interacts with the first moiety of the affinity reagent. In certain embodiments, the functional surface group can interact with the first moiety of the affinity reagent covalently (e.g., a click/SPAAC reaction) or non-covalently (e.g., a charged interaction such as ionic bonding or polar interaction, hydrophobic or van der Waals interactions, a biotin-streptavidin interaction), or a combination thereof. In some examples, the functional surface group comprises a hydrophobic binding group, a negatively charged binding group (e.g., sulfonic, sulfate, phosphoric, phosphonic or carboxylic group), a positively charged binding group (e.g., primary, secondary, tertiary amine and quaternary ammonium, heterocyclic amines, such as pyridine, pyrimidine, pyridinium, piperazine), a polar binding group (e.g., chemical moieties comprising polarized chemical bonds, such C—O, C═O, C—N, C═N, C═N, N—H, O—H, C—F, C—Cl, C—Br, C—S, S—H, S—O, S═O, C—P, P—O, P═O, P—H, more specifically carboxyl, alcohol, thiol, amide, halide, amine, ester, ether, or thioester), or a combination thereof. In certain examples, the functional surface group comprises a hydrophobic binding group selected from an alkyl group, a cycloalkyl group, a haloalkyl group (e.g., fluoroalkyl), an aryl group, or a combination thereof, for interaction with the affinity reagent. In more specific examples, the hydrophobic binding group can comprise a linear alkyl group, optionally wherein the linear alkyl group is selected from a C₄-C₂₀ alkyl group, a C₄-C₁₂ alkyl group, or a C₄-C₁₀ alkyl group. In one embodiment, the functional surface group and the first moiety of the affinity reagent comprises an azide group (e.g., an azidosilane) and a cycloalkyne (e.g., cyclooctyne), and wherein the azido group and the cycloalkyne react to form a nitrogen-containing heterocycle.

The functional surface group can be bound to the solid support any suitable means, such as covalently (e.g., a click/SPAAC reaction) or non-covalently (e.g., a charged interaction such as ionic bonding or polar interaction, hydrophobic or van der Waals interactions, a biotin-streptavidin interaction), or a combination thereof. In some the functional surface group can be bound to the solid support via a triazole group, triazinyl group, an imidazole, an indole, a silane group, a silatrane group, a siloxane group, a cyclic siloxane group, a silsesquioxane group, a silazane group, or a combination thereof. In one embodiments, the functional surface group can be bound to the solid support via a silane group.

The solid support can be derived from any suitable material for capturing cells or nucleic acid during nucleic acid extraction and isolation. In some embodiments, the solid support can be a porous material, such as a fibrous porous material. The porous material can be derived from silica, glass (e.g., glass bead, or glass filter), cellulose (e.g., cellulose filter), ethylenic backbone polymer, mica, polycarbonate (e.g., polycarbonate filter), zeolite, titanium dioxide, magnetic material (e.g., magnetic bead), polyethersulfone (e.g., polyethersulfone filter), polytetrafluoroethylene (e.g., polytetrafluoroethylene filter), polyvinylpyrrolidone (e.g., polyvinylpyrrolidone filter), or a combination. In embodiment, the solid support is a porous material derived from a glass fiber filter. The glass fiber filter can be modified with a functional surface group as described herein, optionally wherein the functional surface group comprises a hydrophobic silane compound.

As described herein, the method comprises contacting a solid support with an affinity reagent and the sample, wherein the affinity reagent comprises a first moiety that interacts with the solid support and a second moiety that interacts with nucleic acid in the sample. The first moiety of the affinity reagent can interact with the solid support by any suitable means, such as covalently (e.g., a click/SPAAC reaction) or non-covalently (e.g., a charged interaction such as ionic bonding or polar interaction, hydrophobic or van der Waals interactions, a biotin-streptavidin interaction), or a combination thereof. In some embodiments, the first moiety of the affinity reagent can interact with the solid support via a hydrophobic group, a negatively charged binding group, a positively charged binding group, a polar group, or a combination thereof. For example, the first moiety of the affinity reagent can comprise a hydrophobic group for interacting with the solid support, wherein the hydrophobic group is selected from an alkyl group, a cycloalkyl group, a haloalkyl group (e.g., fluoroalkyl), an aryl group, or a combination thereof. In some embodiments, the first moiety of the affinity reagent comprises a linear alkyl group, optionally wherein the linear alkyl group is selected from a C₄-C₂₀ alkyl group, a C₄-C₁₂ alkyl group, or a C₄-C₁₀ alkyl group. In some embodiments, the first moiety of the affinity reagent comprises a branched alkyl group, optionally wherein the branched alkyl group is selected from a C₆-C₂₀ alkyl group, a C₈-C₁₂ alkyl group, or a C₈-C₁₀ alkyl group.

The second moiety of the affinity reagent can interact with the nucleic acid by any suitable means, such as covalently (e.g., a click/SPAAC reaction) or non-covalently (e.g., a charged interaction such as ionic bonding or polar interaction, hydrophobic or van der Waals interactions, a biotin-streptavidin interaction), or a combination thereof. For example, the second moiety of the affinity reagent can comprise an amine, a heterocycle (such as triazole, an imidazole, or an indole), an intercalating agent, a DNA groove binder, a peptide, an amino acid, a protein, or a combination thereof, for interaction with the nucleic acid. In one embodiment, the second moiety of the affinity reagent comprise an amine, optionally wherein the amine is selected from an alkylamine, a cycloalkylamine, a branched amine, an alkyloxy amine, a polyamine moiety, an arylamine, or a combination thereof, for interaction with the nucleic acid. In another embodiment, the second moiety of the affinity reagent comprise an alkylamine, an imidazole, a bisbenzimide minor groove binder, polycyclic intercalating agent, or a combination thereof, for interaction with the nucleic acid. In some examples, the second moiety of the affinity reagent comprises spermine, methylamine, ethylamine, propylamine, ethylenediamine, diethylene triamine, 1,3-dimethyldipropylenediamine, 3-(2-aminoethyl)aminopropyl, (2-aminoethyl)trimethylammonium hydrochloride, tris(2-aminoethyl)amine, or a combination thereof, for interaction with the nucleic acid.

The affinity reagent can further comprise a linker that interacts with a solvent and increases solubility of the affinity reagent. In some embodiments, the linker can comprise a hydrophilic group selected from polyethylene glycol linkers, ester-based linkers (such as branched ester-based linkers), amide-based linkers (such as branched amide-based linkers including N,N-dihexyl-3-imidazole-2-oxoamine linkers), amine-based linkers (such as 3-dibutylaminopropylamine linkers, N,N-dihexyl-3-aminopropylamine linkers), organophosphorous-based linkers (such as organophosphine-based linkers, organophosphine oxide-based linkers, organophosphinate-based linkers, organophosphoramidate-based linkers, organophosphate-based linkers, organophosphonamidate-based linkers, or organophosphonate-based linkers), glucuronic acid-based linkers, disulfide linkers, cathepsin B linkers, or combinations thereof.

The affinity reagent can further comprise a photocleavable group for releasing the nucleic acid concentrated on the solid support.

As described in the methods herein, contacting the solid support with the sample and the affinity reagent can be performed under conditions effective to allow interaction between a) the nucleic acid and the affinity reagent and b) the solid support and the affinity reagent. The conditions can include a pH of less than 7. The method can further comprise contacting the sample with a lysis buffer prior to or simultaneously with contacting the solid support with the sample. The lysis buffer can comprise one or more of a chaotropic agent, a salt, a buffering agent, a surfactant, a defoaming agent, and a binding agent.

The method can further comprise releasing the nucleic acid concentrated on the solid support.

The method comprises releasing the nucleic acid concentrated on the solid support, which can be performed in the presence of an eluting agent, heat, sonication, conditions for photochemical cleavage, or a combination thereof. In some embodiments, releasing the nucleic acid concentrated on the solid support can be performed in the presence of an eluting agent, optionally wherein the eluting agent has a pH greater than about 7, greater than about 8, greater than about 9, and/or a salt concentration higher than the sample. In some embodiments, releasing the nucleic acid concentrated on the solid support can be performed in the presence of heat, optionally wherein the nucleic acid, affinity reagent, and solid support are heated to a temperature of 35° C. or greater, 45° C. or greater, 55° C. or greater, 65° C. or greater, 75° C. or greater, 85° C. or greater, 95° C. or greater, or up to 100° C. or greater. In some embodiments, releasing the nucleic acid concentrated on the solid support can be performed in the presence of sonication. In some embodiments, releasing the nucleic acid concentrated on the solid support can be performed in the presence of conditions for photochemical cleavage of the affinity reagent, optionally wherein conditions for photochemical cleavage include exposure of the affinity reagent to UV light.

The methods described herein can further comprise washing the solid support with a wash solution. The wash solution can have a pH of less than 7.

Methods for detecting a nucleic acid in a biological sample are also described. The methods for detecting a nucleic acid in a biological sample can comprise (a) isolating the nucleic acid from a sample using a method as disclosed herein; (b) releasing the nucleic acid from the solid support with an eluting agent; and (c) detecting the nucleic acid.

The methods disclosed can be performed in a cartridge, optionally an automated cartridge. Sample cartridges and automated cartridges are described herein.

Sample cartridge for isolation and detection of nucleic acid from a biological sample can comprise a cartridge body having a plurality of chambers therein, wherein the plurality of chambers includes a sample chamber configured to receive the biological sample; a reaction vessel fluidically coupled to the plurality of chambers and configured for amplification of nucleic acid and detection of a plurality of amplification products; a filter disposed in the fluidic path between the plurality of chambers and the reaction vessel, wherein the filter comprises a solid support having a surface capable of binding an affinity reagent, the affinity reagent disposed in one of the plurality of chambers, the affinity reagent comprising a first moiety that interacts with the solid support and a second moiety that interacts with nucleic acid from the biological sample, and primers and/or probes disposed in one or more chambers of the plurality of chambers or reaction vessel for detection of the nucleic acid. The primers and/or probes may be immobilized on a surface (e.g., a biosensor surface) in one or more chambers of the plurality of chambers or reaction vessel for detection of the nucleic acid. The affinity reagent can further comprise a linker that interacts with a solvent and increases solubility of the affinity reagent.

The plurality of chambers in the sample cartridge can comprise a lysis chamber in fluidic communication with the sample chamber, wherein the lysis chamber comprises lysis reagent for releasing nucleic acid. In some examples, the sample chamber and lysis chamber are the same. The plurality of chambers further comprises a binding reagent, filtering buffer, washing reagent, elution buffer, of combinations thereof.

As described in the sample cartridge, the filter comprises a solid support having a surface capable of binding an affinity reagent. The solid support is as described herein and comprises a functional surface group that interacts with the first moiety of the affinity reagent, optionally, wherein the functional surface group interacts with the first moiety of the affinity reagent covalently (e.g., a click/SPAAC reaction) or non-covalently (e.g., a charged interaction such as ionic bonding or polar interaction, hydrophobic or van der Waals interactions, a biotin-streptavidin interaction), or a combination thereof. The solid support can be porous material such as a glass fiber filter.

The affinity reagent can be present on the solid support withing the cartridge at a surface density of 10 nmoles/cm² or greater, 20 nmoles/cm² or greater, 35 nmoles/cm² or greater, or from 30-100 nmoles/cm², based on the solid support. In some embodiments, the affinity reagent can be present at a surface density of 3,000 nmoles/cm² or less, 2,500 nmoles/cm² or less, 2,000 nmoles/cm² or less, 1,000 nmoles/cm² or less, 500 nmoles/cm² or less, 400 nmoles/cm² or less, 300 nmoles/cm² or less, 200 nmoles/cm² or less, or 100 nmoles/cm² or less, based on the solid support. The solid support together with the affinity reagent has a DNA binding capacity of at least 10 μg/cm², 20 μg/cm² or greater, 35 μg/cm² or greater, or from 30-100 μg/cm².

The solid support within the sample cartridge can have a pore size from 0.2 μm to 3 μm, from 0.2 μm to 2 μm, from 0.5 μm to 1.0 μm, or from 0.6 μm to 0.8 μm. The solid support (e.g., glass fiber filter) can further comprise beads to facilitate mechanical lysis, wherein the beads are selected from glass beads, silica beads, or a combination thereof. The solid support (e.g., glass fiber filter) can have a basis weight from 35 g/m² to 100 g/m², preferably from 50 g/m² to 85 g/m², or from 70 g/m² to 85 g/m². The solid support (e.g., glass fiber filter) can have a fiber diameter from 1 μm to 100 μm, preferably from 1 μm to 50 μm, or from 1 μm to 25 μm. The solid support (e.g., glass fiber filter) can have a thickness from 250 μm to 2,000 μm, from 300 μm to 1,500 μm, from 300 μm to 1,000 μm, from 300 μm to 750 μm, or from 350 μm to 500 μm.

The sample cartridge together with the reagents allow for flow rates up to about 100 μL per second, such as from about 10 μL to about 100 μL. in some embodiments, the sample cartridge together with the reagents allow for pressure below 100 psi, below 80 psi, or below 60 psi. As described herein, the sample cartridge can be an automated cartridge.

Methods for detecting nucleic acid in a biological sample obtained from a subject in the sample cartridge disclosed herein are provided. The method can comprise a) contacting nucleic acid from the biological sample with a set of primers and optional probes in a sample cartridge as disclosed herein; b) subjecting the nucleic acid, primer pairs, and optional probes to amplification conditions; c) detecting the presence of amplification product(s), optionally via real-time PCR, melt curve analysis, or a combination thereof, and d) detecting the presence of the nucleic acid in the biological sample based on detection of the amplification products. In some embodiments, the method does not comprise utilizing a chaotropic agent, a lysis buffer, or a binding agent.

Certain aspects of the present disclosure generally relate to compositions, devices, and methods for determining viruses such as coronaviruses. For instance, some aspects are directed to partitioning a biological sample comprising a virus and free nucleic acid using the devices for isolating and purifying nucleic acid disclosed herein, and determining the virus within the devices. In some cases, at least 95% of the free nucleic acid partitions on the solid support of the devices.

The biological sample can be selected from blood, blood culture, plasma, serum, semen, spinal fluid, tissue, tear, urine, stool, saliva, smear preparation, respiratory sample, nasopharyngeal swab sample, anterior nasal sample, mid-turbinate nasal sample, oropharyngeal sample, vaginal swab, vaginal mucus sample, vaginal tissue sample, vaginal cell sample, bacterial culture, mammalian cell culture, viral culture, human cell, bacteria, extracellular fluid, pancreatic fluid, cell lysate, PCR reaction mixture, or in vitro nucleic acid modification reaction mixture, preferably wherein the biological sample is blood, plasma, respiratory sample, or vaginal swab. In some examples, the biological sample comprises nucleic acid selected from genomic DNA, total RNA, short-DNA, small DNA, tumor-derived nucleic acid, methylated DNA, microbial nucleic acid, bacterial nucleic acid, viral nucleic acid, cell free nucleic acid, or combinations thereof.

Amplification of nucleic acid in the methods and sample cartridges disclosed herein can be via nested PCR, gradient PCR, isothermal PCR, qPCR, or RT-PCR. In one embodiment, amplification is by a real-time PCR multiplex assay.

### BRIEF DESCRIPTION OF THE FIGURES

FIG. 1 is a schematic drawing showing nucleic acid extraction and isolation by a modified solid support in the presence of an affinity reagent.

FIG. 2 is a schematic drawing showing nucleic acid extraction and isolation by a modified solid support in the presence of an affinity reagent comprising a cleavable group.

FIG. 3 is a schematic drawing showing nucleic acid extraction and isolation by a modified solid support in the presence of an affinity reagent and isolation of the nucleic acid by heat.

FIG. 4 is a schematic drawing showing a modified solid support. The solid support is modified with a hydrophobic surface group that is covalently bound to the solid support via a triazole linker.

FIG. 5 shows an overview of a sample cartridge with a reaction vessel (left) and an exploded view of the sample cartridge valve assembly (right).

FIGS. 6A-6C illustrate various valve assemblies and components.

FIGS. 7A-7E illustrate valve assemblies and components, in accordance with some embodiments.

FIGS. 8A-8E illustrate boxplots of measured levels, expressed as a cycle threshold (Ct) value, of nucleic acid from CoV2 virus and free RNA using glass fiber filter (GFF) modified with an amino affinity group (GFF 3) and unmodified glass fiber filter (Reg).

FIGS. 9A and 9B show plots of DNA binding and elution in different buffers and pH as measured by fluorometry.

FIGS. 10A and 10B demonstrate effect of salt on DNA binding and elution as measured by fluorometry.

FIGS. 11A and 11B demonstrate the effect of DNA binding and elution when washed with KCl NaCl, LiCl, NaPi, and GuHCl.

FIG. 12 compares DNA binding and elution with affinity reagent 3DP and DAP.

### DETAILED DESCRIPTION

### Definitions

To facilitate an understanding of the present disclosure, a number of terms and phrases are defined below:

As used in the specification and the appended claims, the singular forms “a,” “an” and “the” include plural referents unless the context clearly dictates otherwise. Thus, for example, reference to “a compound” includes mixtures of compounds, reference to “an amine group” includes mixtures of two or more such amine groups, and the like.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

In this specification and in the claims which follow, reference will be made to a number of terms which shall be defined to have the following meanings:

The term “alkyl” includes both “unsubstituted alkyls” and “substituted alkyls”, the latter of which refers to alkyl moieties having one or more substituents replacing a hydrogen on one or more carbons of the hydrocarbon backbone. Such substituents include, but are not limited to, halogen, hydroxyl, carbonyl (such as a carboxyl, alkoxycarbonyl, formyl, or an acyl), thiocarbonyl (such as a thioester, a thioacetate, or a thioformate), alkoxyl, phosphoryl, phosphate, phosphonate, a phosphinate, amino, amido, amidine, imine, cyano, nitro, azido, sulfhydryl, alkylthio, sulfate, sulfonate, sulfamoyl, sulfonamido, sulfonyl, heterocyclyl, aralkyl, or an aromatic or heteroaromatic moiety.

Unless the number of carbons is otherwise specified, “alkyl” as used herein means an alkyl group, as defined above, but having from one to twenty carbons, more preferably from one to ten carbon atoms in its backbone structure. Likewise, “alkenyl” and “alkynyl” have similar chain lengths.

The alkyl groups can also contain one or more heteroatoms within the carbon backbone. Examples include oxygen, nitrogen, sulfur, and combinations thereof. In certain embodiments, the alkyl group contains between one and four heteroatoms.

The term “heteroalkyl”, as used herein, refers to straight or branched chain, or cyclic carbon-containing radicals, or combinations thereof, containing at least one heteroatom. Suitable heteroatoms include, but are not limited to, O, N, Si, P, Se, B, and S, wherein the phosphorous and sulfur atoms are optionally oxidized, and the nitrogen heteroatom is optionally quaternized. Heteroalkyls can be substituted as defined above for alkyl groups.

“Alkenyl” and “Alkynyl”, as used herein, refer to unsaturated aliphatic groups containing one or more double or triple bonds analogous in length (e.g., C₂-C₃₀) and possible substitution to the alkyl groups described above.

“Aryl”, as used herein, refers to 5-, 6- and 7-membered aromatic rings. The ring can be a carbocyclic, heterocyclic, fused carbocyclic, fused heterocyclic, bicarbocyclic, or biheterocyclic ring system, optionally substituted as described above for alkyl. Broadly defined, “Ar”, as used herein, includes 5-, 6- and 7-membered single-ring aromatic groups that can include from zero to four heteroatoms. Examples include, but are not limited to, benzene, pyrrole, furan, thiophene, imidazole, oxazole, thiazole, triazole, pyrazole, pyridine, pyrazine, pyridazine and pyrimidine. Those aryl groups having heteroatoms in the ring structure can also be referred to as “heteroaryl”, “aryl heterocycles”, or “heteroaromatics”. The aromatic ring can be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, and —CN. The term “Ar” also includes polycyclic ring systems having two or more cyclic rings in which two or more carbons are common to two adjoining rings (the rings are “fused rings”) wherein at least one of the rings is aromatic, e.g., the other cyclic rings can be cycloalkyls, cycloalkenyls, cycloalkynyls, aryls and/or heterocycles, or both rings are aromatic.

“Alkylaryl” or “aryl-alkyl”, as used herein, refers to an alkyl group substituted with an aryl group (e.g., an aromatic or hetero aromatic group).

“Heterocycle” or “heterocyclic”, as used herein, refers to a cyclic radical attached via a ring carbon or nitrogen of a monocyclic or bicyclic ring containing 3-10 ring atoms, and preferably from 5-6 ring atoms, containing carbon and one to four heteroatoms each selected from non-peroxide oxygen, sulfur, and N(Y) wherein Y is absent or is H, O, (C₁₋₄) alkyl, phenyl or benzyl, and optionally containing one or more double or triple bonds, and optionally substituted with one or more substituents. The term “heterocycle” also encompasses substituted and unsubstituted heteroaryl rings. Examples of heterocyclic ring include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl.

“Heteroaryl”, as used herein, refers to a monocyclic aromatic ring containing five or six ring atoms containing carbon and 1, 2, 3, or 4 heteroatoms each selected from non-peroxide oxygen, sulfur, and N(Y) where Y is absent or is H, O, (C₁-C₈) alkyl, phenyl or benzyl. Non-limiting examples of heteroaryl groups include furyl, imidazolyl, triazolyl, triazinyl, oxazoyl, isoxazoyl, thiazolyl, isothiazoyl, pyrazolyl, pyrrolyl, pyrazinyl, tetrazolyl, pyridyl, (or its N-oxide), thienyl, pyrimidinyl (or its N-oxide), indolyl, isoquinolyl (or its N-oxide), quinolyl (or its N-oxide) and the like. The term “heteroaryl” can include radicals of an ortho-fused bicyclic heterocycle of about eight to ten ring atoms derived therefrom, particularly a benz-derivative or one derived by fusing a propylene, trimethylene, or tetramethylene diradical thereto. Examples of heteroaryl include, but are not limited to, furyl, imidazolyl, triazolyl, triazinyl, oxazoyl, isoxazoyl, thiazolyl, isothiazoyl, pyraxolyl, pyrrolyl, pyrazinyl, tetrazolyl, pyridyl (or its N-oxide), thientyl, pyrimidinyl (or its N-oxide), indolyl, isoquinolyl (or its N-oxide), quinolyl (or its N-oxide), and the like.

The terms “alkoxyl” or “alkoxy” as used herein refers to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, and —O-alkynyl. Aroxy can be represented by —O-aryl or O-heteroaryl, wherein aryl and heteroaryl are as defined below. The alkoxy and aroxy groups can be substituted as described above for alkyl.

The terms “amine” and “amino” are art-recognized and refer to both unsubstituted and substituted amines, e.g., a moiety that can be represented by the general formula: —NR₉R₁₀ or NR₉R₁₀R′₁₀, wherein R₉, R₁₀, and R′₁₀ each independently represent a hydrogen, an alkyl, an alkenyl, —(CH₂)ₘ—R'₈ or R₉ and R₁₀ taken together with the N atom to which they are attached complete a heterocycle having from 4 to 8 atoms in the ring structure; R's represents an aryl, a cycloalkyl, a cycloalkenyl, a heterocycle or a polycycle; and m is zero or an integer in the range of 1 to 8. In some embodiments, only one of R₉ or R₁₀ can be a carbonyl, e.g., R₉, R₁₀ and the nitrogen together do not form an imide. In some embodiments, the term “amine” does not encompass amides, e.g., wherein one of R₉ and R₁₀ represents a carbonyl. In some embodiments, R₉ and R₁₀ (and optionally R′₁₀) each independently represent a hydrogen, an alkyl or cycloakly, an alkenyl or cycloalkenyl, or alkynyl. Thus, the term “alkylamine” as used herein means an amine group, as defined above, having a substituted (as described above for alkyl) or unsubstituted alkyl attached thereto, i.e., at least one of R₉ and R₁₀ is an alkyl group.

The terms “amido” or “amide” is art-recognized as an amino-substituted carbonyl and includes a moiety that can be represented by the general formula-CONR₉R₁₀ wherein R₉ and R₁₀ are as defined above.

“Halogen”, as used herein, refers to fluorine, chlorine, bromine, or iodine.

“Hydroxyl”, as used herein, refers to —OH.

The term “carbonyl” is art-recognized and includes such moieties as can be represented by the general formula —CO—XR₁₁, or —X—CO—R′₁₁, wherein X is a bond or represents an oxygen or a sulfur, and R₁₁ represents a hydrogen, an alkyl, a cycloalkyl, an alkenyl, a cycloalkenyl, or an alkynyl, R′₁₁ represents a hydrogen, an alkyl, a cycloalkyl, an alkenyl, a cycloalkenyl, or an alkynyl. Where X is an oxygen and R₁₁ or R′₁₁ is not hydrogen, the formula represents an “ester”. Where X is an oxygen and Ru is as defined above, the moiety is referred to herein as a carboxyl group, and particularly when Rn is a hydrogen, the formula represents a “carboxylic acid”. Where X is an oxygen and R′n is hydrogen, the formula represents a “formate”. In general, where the oxygen atom of the above formula is replaced by sulfur, the formula represents a “thiocarbonyl” group. Where X is a sulfur and R₁₁ or R′₁₁ is not hydrogen, the formula represents a “thioester.” Where X is a sulfur and R₁₁ is hydrogen, the formula represents a “thiocarboxylic acid.” Where X is a sulfur and R′n is hydrogen, the formula represents a “thioformate.” On the other hand, where X is a bond, and Ru is not hydrogen, the above formula represents a “ketone” group. Where X is a bond, and Rn is hydrogen, the above formula represents an “aldehyde” group.

The term “substituted” as used herein, refers to all permissible substituents of the compounds described herein. In the broadest sense, the permissible substituents include acyclic and cyclic, branched and unbranched, carbocyclic and heterocyclic, aromatic and nonaromatic substituents of organic compounds. Illustrative substituents include, but are not limited to, halogens, hydroxyl groups, or any other organic groupings containing any number of carbon atoms, preferably 1-14 carbon atoms, and optionally include one or more heteroatoms such as oxygen, sulfur, or nitrogen grouping in linear, branched, or cyclic structural formats. Representative substituents include alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aryloxy, substituted aryloxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, aminoacid, peptide, and polypeptide groups.

It is understood that “substitution” or “substituted” includes the implicit proviso that such substitution is in accordance with permitted valence of the substituted atom and the substituent, and that the substitution results in a stable compound, i.e. a compound that does not spontaneously undergo transformation such as by rearrangement, cyclization, elimination, etc.

As used herein, the terms “hydrolyzable” refers to a group or moiety which is capable of undergoing hydrolysis or solvolysis. For example, a hydrolyzable group can be hydrolyzed (i.e., converted to a hydrogen group) by exposure to water or a protic solvent at or near ambient temperature or an elevated temperature and at or near atmospheric pressure or an elevated pressure. In some cases, a hydrolyzable group can be hydrolyzed by exposure to acidic or alkaline water or acidic or alkaline protic solvent. Typical hydrolyzable groups include, but are not limited to, alkoxy, aryloxy, aralkyloxy, acyloxy, or halo. As used herein, the term is often used in reference to one of more groups bonded to a silicon atom in a silyl group.

As used herein, the terms “detect”, “detecting” or “detection” may describe either the general act of discovering or discerning or the specific observation of a detectably labeled composition.

The term “nucleic acid” refers to a nucleotide polymer, and unless otherwise limited, includes analogs of natural nucleotides that can function in a similar manner (e.g., hybridize) to naturally occurring nucleotides. The term nucleic acid includes any form of DNA or RNA, including, for example, genomic DNA; complementary DNA (cDNA), which is a DNA representation of mRNA, usually obtained by reverse transcription of messenger RNA (mRNA) or viral RNA or by amplification; DNA molecules produced synthetically or by amplification; mRNA; and non-coding RNA. The nucleic acid can be of any origin, including mammalian (e.g. human), bacterial, viral, or synthetic origin. The term nucleic acid encompasses double- or triple-stranded nucleic acid complexes, as well as single-stranded molecules. In double- or triple-stranded nucleic acid complexes, the nucleic acid strands need not be coextensive (i.e, a double-stranded nucleic acid need not be double-stranded along the entire length of both strands).

The term nucleic acid also encompasses any modifications thereof, such as by methylation and/or by capping. Nucleic acid modifications can include addition of chemical groups that incorporate additional charge, polarizability, hydrogen bonding, electrostatic interaction, and functionality to the individual nucleic acid bases or to the nucleic acid as a whole. Such modifications may include base modifications such as 2′-position sugar modifications, 5-position pyrimidine modifications, 8-position purine modifications, modifications at cytosine exocyclic amines, substitutions of 5-bromo-uracil, sugar-phosphate backbone modifications, unusual base pairing combinations such as the isobases isocytidine and isoguanidine, and the like. More particularly, in some embodiments, nucleic acids, can include polydeoxyribonucleotides (containing 2-deoxy-D-ribose), polyribonucleotides (containing D-ribose), and any other type of nucleic acid that is an N- or C-glycoside of a purine or pyrimidine base, as well as other polymers containing nonnucleotidic backbones, for example, polyamide (e.g., peptide nucleic acids (PNAs)) and polymorpholino polymers (see, e.g., Summerton and Weller (1997) “Morpholino Antisense Oligomers: Design, Preparation, and Properties,” Antisense & Nucleic Acid Drug Dev. 7:1817-195; Okamoto et al. (2002) “Development of electrochemically gene-analyzing method using DNA-modified electrodes,” Nucleic Acids Res. Supplement No. 2:171-172), and other synthetic sequence-specific nucleic acid polymers providing that the polymers contain nucleobases in a configuration which allows for base pairing and base stacking, such as is found in DNA and RNA. The term nucleic acid also encompasses locked nucleic acids (LNAs), which are described in U.S. Pat. Nos. 6,794,499, 6,670,461, 6,262,490, and 6,770,748, which are incorporated herein by reference in their entirety for their disclosure of LNAs. The nucleic acid(s) can be derived from a completely chemical synthesis process, such as a solid phase-mediated chemical synthesis, from a biological source, such as through isolation from any species that produces nucleic acid, or from processes that involve the manipulation of nucleic acids by molecular biology tools, such as DNA replication, PCR amplification, reverse transcription, or from a combination of those processes.

As used herein, the terms “oligonucleotide,” “polynucleotide,” and the like, refer to nucleic acid-containing molecules, including but not limited to, DNA or RNA. The term “oligonucleotide” is used to refer to a nucleic acid that is relatively short, generally shorter than 500 nucleotides, particularly, shorter than 200 nucleotides, more particularly, shorter than 100 nucleotides, most particularly, shorter than 50 nucleotides. Typically, oligonucleotides are single-stranded DNA molecules. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

In some embodiments, an oligonucleotide can range from about 8 to 200, 8 to 100, 12 to 200, 12 to 100, 12 to 75, or 12 to 50 nucleotides long. Oligonucleotides may be referred to by their length, for example, a 24 residue oligonucleotide may be referred to as a “24-mer.”

The term “nucleic acid amplification,” encompasses any means by which at least a part of at least one target nucleic acid is reproduced, typically in a template-dependent manner, including without limitation, a broad range of techniques for amplifying nucleic acid sequences, either linearly or exponentially. Exemplary means for performing an amplifying step include polymerase chain reaction (PCR), ligase chain reaction (LCR), ligase detection reaction (LDR), multiplex ligation-dependent probe amplification (MLPA), ligation followed by Q-replicase amplification, primer extension, strand displacement amplification (SDA), hyperbranched strand displacement amplification, multiple displacement amplification (MDA), nucleic acid strand-based amplification (NASBA), two-step multiplexed amplifications, rolling circle amplification (RCA), recombinase polymerase amplification and the like, including multiplex versions and combinations thereof, for example but not limited to, OLA/PCR, PCR/OLA, LDR/PCR, PCR/PCR/LDR, PCR/LDR, LCR/PCR, PCR/LCR (also known as combined chain reaction—CCR), digital amplification, and the like. Descriptions of such techniques can be found in, among other sources, Ausbel et al.; PCR Primer: A Laboratory Manual, Diffenbach, Ed., Cold Spring Harbor Press (1995); U.S. Pat. Nos. 5,830,711, 6,027,889, and 5,686,243, each of which are incorporated herein in their entirety.

A “sample,” or “biological sample” as used herein, includes various nucleic acid (e.g., DNA and/or RNA) containing samples of tissue, cells, or fluid isolated from a subject, including but not limited to, for example, whole blood, buffy coat, plasma, serum, immune cells (e.g., monocytes or macrophages), blood cultures, swabs, saliva, and sputa. In some embodiments, the sample comprises a buffer, such as an anticoagulant, and/or a preservative. In some embodiments whole blood is mixed with heparin in a lithium heparin blood collection tube. The sample can be from any bodily fluid, tissue or cells that contain the expressed biomarker. A biological sample can be obtained from a subject by conventional techniques. For example, blood can be obtained by venipuncture or a finger-prick capillary, and solid tissue samples can be obtained by surgical techniques according to methods well known in the art. In some aspects the blood sample is placed into a tube that is specifically designed for the assay. In some aspects, the sample is a nasal swab. For example, the swab is obtained fresh or stored in a transport medium such as UTM or VTM.

A “reagent” refers broadly to any agent used in a reaction, other than the analyte (e.g., nucleic acid being analyzed). Illustrative reagents for a nucleic acid amplification reaction include, but are not limited to, an affinity reagent, buffer, metal ions, polymerase, reverse transcriptase, primers, template nucleic acid, nucleotides, labels, dyes, nucleases, dNTPs, and the like. Reagents for enzyme reactions include, for example, substrates, cofactors, buffer, metal ions, inhibitors, and activators.

As used herein, the term “detecting” refers to “determining the presence of” an item, such as a nucleic acid sequence, e.g., one that is indicative of the presence of a virus.

As used herein, “Clinical Laboratory Improvement Amendments (CLIA)” refers to The Clinical Laboratory Improvement Amendments of 1988 (CLIA) regulations in effect as of the original filing date of the present application. The CLIA regulations include federal standards applicable to all U.S. facilities or sites that test human specimens for health assessment or to diagnose, prevent, or treat disease. A “CLIA-compliant” test is one that complies with these regulations. “CLIA-waived” tests include tests that does not comply with all of these regulations. For example, CLIA-waived tests include test systems cleared by the U.S. Food and Drug Administration for home use and those tests approved for waiver under the CLIA criteria.

### Solid Support

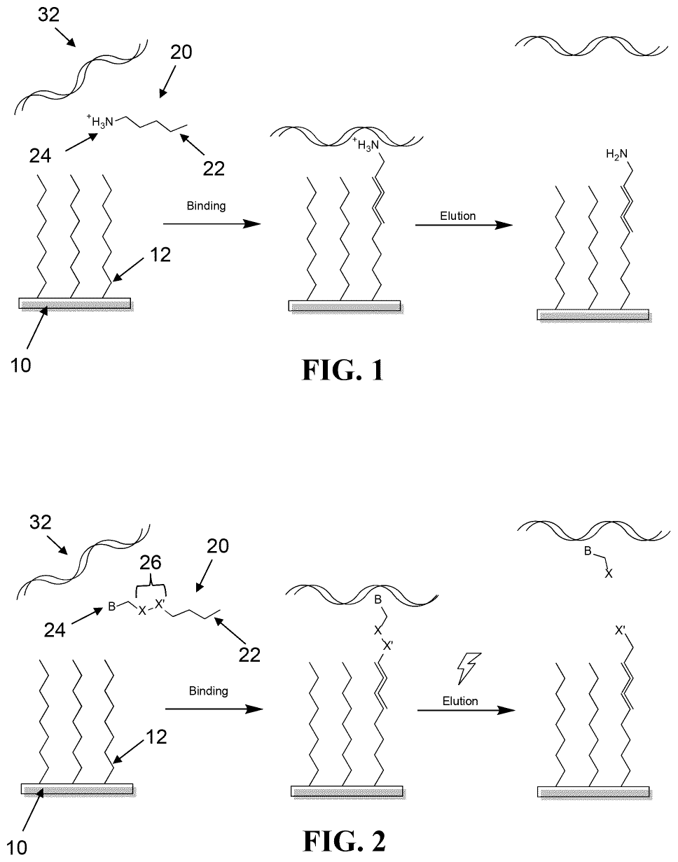

Provided herein are solid support materials used in isolation and purification of nucleic acids from nucleic acid-containing samples. FIG. 1 illustrates an exemplary embodiment of nucleic acid extraction and isolation by a modified solid support 10 in the presence of an affinity reagent 20. In some embodiments, the solid support 10 can be modified with a functional surface group 12 that binds to or interact with an affinity reagent described herein, to render the surface selective towards the interaction with nucleic acids. In some examples, the functional surface group is chemically bonded to a surface of the solid support. FIG. 1 shows a solid support 10 modified with a hydrophobic surface group 12 that can interact with at least a portion of the affinity reagent. For example, the functional surface group can be covalently bonded (such as via a triazole linker, as shown in FIG. 4) to the solid support. In other examples, the functional surface group is electrostatically bonded to the solid support or interacts with the solid support via polar, van der Waals, and/or hydrophobic interaction. In other embodiments, the solid support binds to or interact with an affinity reagent to render the surface selective towards the interaction with nucleic acids. In the disclosure provided herein, the solid support generally includes surface functional groups that can interact and/or reactive with the affinity reagent.

The solid support can be any substrate suitable for interacting with biomolecules and can include, without limitation, paramagnetic particles, gels, fibers, controlled pore glass, magnetic beads, microspheres, nanospheres, capillaries, filter membranes, columns, cloths, wipes, paper, flat supports, multi-well plates, porous membranes, porous monoliths, wafers, combs, or any combination thereof. Preferably, the solid support is a porous material and can comprise any suitable material, including but not limited to glass, silica, titanium oxide, aluminum oxide, iron oxide, ethylenic backbone polymers, polypropylene, polyethylene, polystyrene, ceramic, cellulose, nitrocellulose, magnetic silica particles (such as MagneSil™ particles available from Promega Corporation), and divinylbenzene. In some embodiments, the solid support comprises a material selected from polystyrene, glass, ceramic, polypropylene, polyethylene, silica, mica, titanium dioxide, polycarbonate, latex, PMMA, zeolite, polyethersulfone, carboxymethylcellulose, cellulose, and combinations thereof. Examples of solid support includes a magnetic bead, a glass bead, a polystyrene bead, cellulose filter, a polystyrene filter, a polycarbonate filter, a polyethersulfone filter, polytetrafluoroethylene filter, polyvinylpyrrolidone filter, or a glass fiber filter. The solid support may further comprise a polymeric binder for binding the particles or fibers in the solid support. Exemplary polymeric binders include an acrylic polymer.

In some embodiments, the solid support is a fiber material, preferably, a glass fiber filter (GFF). Fibrous filters such as glass fiber filters offer several advantages over other porous supports such as glass beads. Porous glass fiber filters have much larger surface area than flat glass surfaces, but less than porous glass beads. Unlike glass beads, the thin, paper-like sheets of glass fiber filters are easily handled in aqueous or organic solvents. Mechanical stability of the glass fiber filters allows belts and sheets to be used in high throughput manufacturing. Unlike beads, glass fiber filter simplifies flow through filtering with no containing frits required. This feature of GFF allows construction of multilayer devices, where several modified glass filters can be stacked on each other inside a cylindrical, flow through housing. If more DNA binding capacity is required, the effective thickness of the filters can be adjusted by stacking multiple discs.

The solid support preferably has a relatively high surface area to enable high binding capacity of the nucleic acids. When the solid support is a fiber material, the fiber material can be characterized by fiber diameter, pore diameter, basis weight, thickness, and/or specific surface area. The fibers in the fiber material can have an average diameter of 1 micron or greater, 1.5 microns or greater, 2 microns or greater, 2.5 microns or greater, 3 microns or greater, 3.5 microns or greater, 4 microns or greater, 4.5 microns or greater, 5 microns or greater, 5.5 microns or greater, 6 microns or greater, 6.5 microns or greater, 7 microns or greater, 7.5 microns or greater, 8 microns or greater, 9 microns or greater, 10 microns or greater, 12 microns or greater, 15 microns or greater, 16 microns or greater, 18 microns or greater, 19 microns or greater, or 20 microns or greater, or any value in between (e.g., 11.4 microns). In certain embodiments, the fibers in the fiber material can have an average diameter of 25 microns or less, 24 microns or less, 22 microns or less, 20 microns or less, 19 microns or less, 18 microns or less, 16 microns or less, 15 microns or less, 14 microns or less, 13 microns or less, 12 microns or less, 10 microns or less, 9 microns or less, 8 microns or less, 7.5 microns or less, 7 microns or less, 6 microns or less, 5.5 microns or less, or 5 microns or less, or any value in between (e.g., 17.1 microns). In certain embodiments, the fibers in the fiber material can have an average diameter from 1 micron to 20 microns, from 2 microns to 20 microns, from 2 microns to 18 microns, from 2.5 microns to 15 microns, from 2.5 microns to 12 microns, from 2.5 microns to 10 microns, from 3 microns to 20 microns, from 3 microns to 18 microns, from 3 microns to 15 microns, from 3 microns to 12 microns, from 4 microns to 20 microns, from 4 microns to 15 microns, or from 5 microns to 20 microns.

The fiber material can have an effective pore size (or average pore diameter) of 0.20 microns or greater, 0.30 microns or greater, 0.40 microns or greater, 0.50 microns or greater, 0.55 microns or greater, 0.60 microns or greater, 0.65 microns or greater, 0.70 microns or greater, 0.75 microns or greater, 0.80 microns or greater, 0.85 microns or greater, 0.90 microns or greater, 0.95 microns or greater, 1.0 microns or greater, or 1.1 microns or greater, or any value in between (e.g., 0.511 microns). In certain embodiments, the fiber material can have an average pore size of 3.0 microns or less, 2.0 microns or less, 1.9 microns or less, 1.8 microns or less, 1.7 microns or less, 1.6 microns or less, 1.5 microns or less, 1.4 microns or less, 1.3 microns or less, 1.2 microns or less, 1.1 microns or less, 1.05 microns or less, 1.0 microns or less, 0.95 microns or less, 0.90 microns or less, 0.85 microns or less, 0.80 microns or less, 0.75 microns or less, or 0.70 microns or less, or any value in between (e.g., 1.67 microns). In certain embodiments, the fiber material can have an average pore size from 0.2 μm to 3 μm, from 0.20 microns to 2.0 microns, from 0.20 microns to 1.5 microns, from 0.40 microns to 1.5 microns, from 0.40 microns to 1.3 microns, from 0.40 microns to 1.2 microns, from 0.50 microns to 1.5 microns, from 0.50 microns to 1.3 microns, from 0.50 microns to 1.2 microns, from 0.60 microns to 1.5 microns, from 0.60 microns to 1.3 microns, from 0.60 microns to 1.2 microns, from 0.70 microns to 1.5 microns, from 0.70 microns to 1.3 microns, from 0.70 microns to 1.2 microns, or from 0.70 microns to 1.0 micron.

The fiber material, such as the glass fiber filter can have a pore size selected to accommodate correspondingly sized beads to facilitate mechanical lysis. The beads can include glass beads, silica beads, or a combination thereof.

The thickness of the fiber material can be 100 microns or greater, 150 microns or greater, 200 microns or greater, 250 microns or greater, 300 microns or greater, 350 microns or greater, 400 microns or greater, 450 microns or greater, 500 microns or greater, 550 microns or greater, 600 microns or greater, 650 microns or greater, 700 microns or greater, 750 microns or greater, 800 microns or greater, 900 microns or greater, 1,000 microns or greater, 1,200 microns or greater, 1,500 microns or greater, 1,600 microns or greater, 1,800 microns or greater, 1,900 microns or greater, or 2,000 microns or greater, or any value in between (e.g., 1,709 microns). In certain embodiments, the fiber material can have a thickness of 2,500 microns or less, 2,400 microns or less, 2,200 microns or less, 2,000 microns or less, 1,900 microns or less, 1,800 microns or less, 1,600 microns or less, 1,500 microns or less, 1,400 microns or less, 1,300 microns or less, 1,200 microns or less, 1,000 microns or less, 900 microns or less, 800 microns or less, 750 microns or less, 700 microns or less, 600 microns or less, 550 microns or less, or 500 microns or less, or any value in between (e.g., 867.1 microns). In certain embodiments, the fiber material can have a thickness from 100 microns to 2,000 microns, from 200 microns to 1,500 microns, from 200 microns to 1,200 microns, from 250 microns to 1,200 microns, from 250 microns to 1,000 microns, from 250 microns to 800 microns, from 300 microns to 2,000 microns, from 300 microns to 1,800 microns, from 300 microns to 1,500 microns, from 300 microns to 1,200 microns, from 400 microns to 2,000 microns, from 400 microns to 1,500 microns, or from 500 microns to 2,000 microns.

The basis weight of the fiber material can be 10 g/m² or greater, 15 g/m² or greater, 20 g/m² or greater, 25 g/m² or greater, 30 g/m² or greater, 35 g/m² or greater, 40 g/m² or greater, 45 g/m² or greater, 50 g/m² or greater, 55 g/m² or greater, 60 g/m² or greater, 65 g/m² or greater, 70 g/m² or greater, 75 g/m² or greater, 80 g/m² or greater, 90 g/m² or greater, 100 g/m² or greater, 120 g/m² or greater, 150 g/m² or greater, 160 g/m² or greater, 180 g/m² or greater, 190 g/m² or greater, or 200 g/m² or greater, or any value in between (e.g., 58 g/m²). In certain embodiments, the fiber material can have a basis weight of 250 g/m² or less, 240 g/m² or less, 220 g/m² or less, 200 g/m² or less, 190 g/m² or less, 180 g/m² or less, 160 g/m² or less, 150 g/m² or less, 140 g/m² or less, 130 g/m² or less, 120 g/m² or less, 100 g/m² or less, 90 g/m² or less, 80 g/m² or less, 75 g/m² or less, 70 g/m² or less, 60 g/m² or less, 55 g/m² or less, or 50 g/m² or less, or any value in between (e.g., 182 g/m²). In certain embodiments, the fiber material can have a basis weight from 10 g/m² to 200 g/m², from 20 g/m² to 150 g/m², from 20 g/m² to 120 g/m², from 25 g/m² to 120 g/m², from 25 g/m² to 100 g/m², from 25 g/m² to 90 g/m², from 30 g/m² to 200 g/m², from 30 g/m² to 180 g/m², from 30 g/m² to 150 g/m², from 30 g/m² to 100 g/m², from 40 g/m² to 150 g/m², from 40 g/m² to 100 g/m², or from 50 g/m² to 90 g/m².

In some examples, unmodified solid support (which can be modified to include a functional surface group) can be obtained from Pall Corporation, having different pore sizes and thickness: For example, glass fiber filters are available from Pall Corporation as Type A/E, A/B and A/C, all have 1 μm nominal pore size with thickness of 0.33 mm, 0.66 mm and 0.25 mm respectively. Others filter types are described below. The Pall TCLP (Toxic Characteristics Leaching Procedure) product has the same dimensions as the Whatman (Cytiva) filters (0.7 μm pore size, 0.4 mm thick). Specifications of exemplary glass fiber filters include borosilicate glass without binder having a nominal pore size of 3 μm and a thickness of 660 μm (26 mils); glass fiber with acrylic binder having a nominal pore size of 1 μm and a thickness of 1270 μm (50 mils); ultrafine glass fiber with acrylic binder having a nominal pore size of 0.5 μm and a thickness of 330 μm (13 mils); and borosilicate glass without binder having a nominal pore size of 0.7 μm and a thickness of 432 μm (17 mils). In some embodiments, the solid support is a glass fiber filter having a thickness of from 400 microns to 2000 microns and a pore size of 0.5 microns to 1 micron.

In other embodiments, the solid support is particulate material, such as silica gel or silica having a pore diameter from about 30 to about 1000 Angstroms, a particle size from about 2 to about 300 microns, and a specific surface area from about 35 m²/g to about 1000 m²/g. In some embodiments, particulate material can have a pore diameter of about 40 Angstroms to about 500 Angstroms, about 60 Angstroms to about 500 Angstroms, about 100 Angstroms to about 300 Angstroms, and about 150 Angstroms to about 500 Angstroms. In some embodiments, the particulate material can have a particle size of about 2 to about 25 microns, about 5 to about 25 microns, about 15 microns, about 63 to about 200 microns, and about 75 to about 200 microns; and a specific surface area of about 100 m²/g to about 350 m²/g, about 100 m²/g to about 500 m²/g, about 65 m²/g to about 550 m²/g, about 100 m²/g to about 675 m²/g, and about 35 to about 750 m²/g.

As described herein, the solid support can comprise a functional surface group that interacts with and/or is reactive with an affinity reagent. The functional surface group can comprise a functional group such as a silanizing group or another moiety that can facilitate binding with the solid support. For example, the functional surface group can comprise an ether, a silyl ether, a siloxane, an ester of carboxylic acid, an ester of sulfonic acid, an esters of sulfamic acid, an ester of sulfuric acid, an ester of phosphonic acid, an ester of phosphinic acid, an ester of phosphoric acid, a silyl ester of carboxylic acid, a silyl ester of sulfonic acid, a silyl ester of sulfinic acid, a silyl ester of sulfuric acid, a silyl ester of phosphonic acid, a silyl ester of phosphinic acid, a silyl ester of phosphoric acid, an oxides, a sulfide, a carbocycle, a heterocycle with at least one oxygen atom, a heterocycle with at least one nitrogen atom, a heterocycle with at least one sulfur atom, a heterocycle with at least one silicon atom, a carbodiimide (such as DCC and EDCI), a phosphonium or imonium (such as BOP, PyBOP, PyBrOP, TBTU, HBTU, HATU, COMU, and TFFH), a ‘click’ reaction-derived heterocycle, a Diels-Alder reaction-derived carbocycle, a Diels-Alder reaction-derived heterocycle, an amide, an imide, a sulfide, a thiolate, a metal thiolate, a urethane, an oxime, a hydrazide, a hydrazone, a physisorbed or chemisorbed or otherwise non-covalently attached moiety, or a combination thereof. In certain embodiments, the functional surface group includes a functional group selected from a maleimide, an acrylate, an acrylamide, an epoxide, an aziridine, a thiirane, an aldehyde, a ketone, an azide, an alkyne, a disulfide, an anhydride, a carboxylates phosphate, a phosphonate, a sulfate, a sulfonate, a nitrate, an amidine, a silane, a siloxane (e.g., a cyclic siloxane), a triazinyl, a silatrane, a silsesquioxane, a silazane, a cyanate, an acetylene, a cyanide, a halogen, an acetal, a ketal, an amino, carbonyl, a carboxyl, biotin, cyclodextrin, an adamantane, or a vinyl group that can be attached to the solid support.

The functional surface group can also comprise a functional binding group that facilitates binding with the affinity reagent. For example, the functional surface group can comprise a hydrophobic binding moiety, a negatively charged binding group (e.g., sulfonic, sulfate, phosphoric, phosphonic or carboxylic group), a positively charged binding group (e.g., primary, secondary, tertiary amine and quaternary ammonium, heterocyclic amines, such as pyridine, pyrimidine, pyridinium, piperazine), a polar binding moiety (e.g., chemical moieties comprising polarized chemical bonds, such C—O, C═O, C—N, C═N, C═N, N—H, O—H, C—F, C—Cl, C—Br, C—S, S—H, S—O, S═O, C—P, P—O, P—O, P—H, more specifically carboxyl, alcohol, thiol, amide, halide, amine, ester, ether, or thioester), or a combination thereof, that binds with the affinity reagent.

In some examples, the functional surface group is a silane. Silanes comprise at least one silicon atom with four bonds to other atoms such as carbon, hydrogen, oxygen, and the like. In specific examples, the functional surface group can be a silane comprising one or more alkyl, heteroalkyl, cycloalkyl, cycloheteroalkyl, alkenyl, cycloalkenyl, alkynyl, aryl, heteroaryl, haloalkyl, or derivatives thereof. In other specific examples, the functional surface group can comprise a silane having one or more alkoxy groups, such as a methoxy silane, a dimethoxy silane, a trimethoxy silane, an ethoxy silane, a diethoxy silane, a triethoxy silane, a chlorosilane, or derivatives thereof. Silanes comprising alkoxy groups as described herein can crosslink with other silanes on the solid support. In further examples, the silane can include a hydrophobic group such as a linear alkyl group. Linear alkyl groups include, for example, a C₄-C₂₀ alkyl group, a C₄-C₁₂ alkyl group, or a C₄-C₁₀ alkyl group. The silane can comprise a dipodal silane. Alternatively, or in addition thereto, the functional surface can include branched alkyl groups, such as, for example, a C₆-C₂₀ alkyl group, a C₈-C₁₂ alkyl group, or a C₈-C₁₀ alkyl group. In some embodiments, the functional surface group is a compound having the formula:

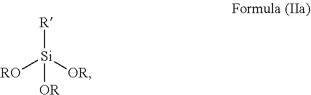

wherein R′ is a moiety that interacts with and/or reactive with the affinity reagent and R is a moiety that interacts with and/or reactive with the solid-support; or wherein R is a moiety that interacts with and/or reactive with the affinity reagent and R′ is a moiety that interacts with and/or reactive with the solid-support. In some examples of R or R′, each can be defined as a group-L-NM, where L is a linker and NM is a moiety that binds to an affinity reagent or a nucleic acid. The linker can be selected from an alkyleneoxy (e.g., a C₂-C₄ alkyleneoxy) group, an alkylene (e.g., a C₂-C₄ alkylene or C₂-C₃ alkylene) group, or a heteroalkylene (e.g., C₄-C₆ heteroalkylene). In some examples, the linker is a bond.

In some embodiments for Formula IIa, R′ is a group-L-NM, where L is a linker and NM is a nucleic acid binding moiety. For example, NM can be an amine group or an intercalator such as pyrene or a pyrene derivative. In some embodiments, the functional surface group can be selected from (3-aminopropyl)triethoxysilane, (3-aminopropyl)trimethoxysilane, N-(6-aminohexyl)aminomethyltriethoxysilane, or N-(2-aminoethyl)-3-aminopropyltrimethoxysilane.

In some examples for Formula IIa, R′ is alkyl or fluoroalkyl. For example, the functional surface group can be a compound having the formula:

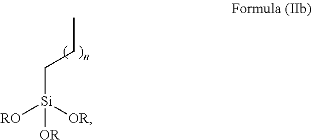

wherein R is an alkyl group. In some examples of Formula IIb, the functional surface group is a trimethoxyalkylsilane or a triethoxyalkylsilane. For example, the functional surface group can be selected from trimethoxyhexylsilane, trimethoxyheptylsilane, trimethoxyoctylsilane, trimethoxynonylsilane, trimethoxydecylsilane, trimethoxyundecylsilane, trimethoxydodecylsilane, trimethoxytridecylsilane, trimethoxytetradecylsilane, trimethoxypentadecylsilane, trimethoxyhexadecylsilane, trimethoxyheptadecylsilane, or trimethoxyoctadecylsilane. In other examples, the functional surface group is triethoxyhexylsilane, triethoxyheptylsilane, triethoxyoctylsilane, triethoxynonylsilane, triethoxydecylsilane, triethoxyundecylsilane, triethoxydodecylsilane, triethoxytridecylsilane, triethoxytetradecylsilane, triethoxypentadecylsilane, triethoxyhexadecylsilane, triethoxyheptadecylsilane, or triethoxyoctadecylsilane.

In some embodiments, the functional surface group can be selected from a fluorinated silane such as heptadecafluorodecyltrimethoxysilane, heptadecafluorodecyltriethoxysilane, heptadecafluorononadecyltrimethoxysilane, heptadecafluorononadecyltriethoxysilane, tridecafluoroheptadecyltrimethoxysilane, tridecafluoroheptadecyltriethoxysilane, nonafluoropentadecyltrimethoxysilane, nonafluoropentadecyltriethoxysilane, nonafluorohexyltrimethoxysilane, nonafluorohexyltriethoxysilane, tridecafluorooctyltrimethoxysilane, tridecafluorooctyltriethoxysilane, or a pentafluoroaryl group such as pentafluorophenoxyundecyltrimethoxysilane.

In some aspects, the surface of the solid support comprises an azido group. Functionalization of the azido group can be performed by reaction with an alkyne-containing functional surface group. For example, the functional surface group can react with an alkyne-containing group having the formula: A-L-NM, where “A” is an alkyne, “L” is a linker, and “NM” is an affinity reagent binding moiety or nucleic acid binding moiety. In some examples, the alkyne is a constrained alkyne such as diazabicyclooctyne (DBCO). In some embodiments, the alkyne is a DBCO group and NM is a hydrophobic group such as an alkyl group.

In some embodiments, the solid support is glass comprising silanol and siloxane groups. Such silanol groups on the glass surface can be reacted with silane- and siloxane-containing functional surface groups to provide a surface having a compound chemically bonded via a siloxane bridge.

Functionalization of the solid support may be performed by any method known in the art. For example, the functionalization may be performed by silanation. Silanation may be performed, for example, in liquid solution or in gas phase (e.g. Physical Vapor Deposition (PVD), Chemical Vapor Deposition (CVD), Atomic Layer Deposition (ALD), and the like).

In some examples, functionalization of the solid support is by deposition which may comprise one or more CVD steps. CVD can comprise loading the substrate into a vacuum oven, loading the silane to be deposited, pumping down the vacuum of the oven, allowing the substrate to dwell, and optionally performing a water contact angle (WCA) test to measure hydrophobicity of the surface. A deposition may be repeated more than once. For example, a deposition step may be repeated 1, 2, 3, 4, 5, 6, 7, 8, 9, 10, or more times. The substrate may be allowed to dwell in the oven for at least about 5 minutes, 10 minutes, 15 minutes, 20 minutes, 25 minutes, 30 minutes, 35 minutes, 40 minutes, 45 minutes, 50 minutes, 55 minutes, 60 minutes, 65 minutes, 70 minutes, 75 minutes, 80 minutes, 85 minutes, 90 minutes, 95 minutes, 100 minutes, 105 minutes, 110 minutes, 115 minutes, 120 minutes, or longer. The conditions in the oven may comprise a reduced pressure atmosphere. For example, the pressure in the oven may be about 1 torr, 2 torr, 3 torr, 4 torr, 5 torr, 10 torr, 15 torr, 20 torr, 50 torr, 100 torr, 200 torr, 500 torr, or less. Alternatively or additionally, the conditions in the oven may comprise a certain temperature. The temperature in the oven may be at least about 50° C., 60° C., 70° C., 80° C., 90° C., 100° C., 110° C., 120° C., 130° C., 140° C., 150° C. or more.

The density of the functional surface group can be controlled by either deposition via liquid or vapor phase methods. In some embodiments, the density of the functional surface group (and the affinity reagent) is 1 nmoles/cm² or greater (for e.g., 5 nmoles/cm² or greater, 10 nmoles/cm² or greater, 15 nmoles/cm² or greater, 20 nmoles/cm² or greater, 25 nmoles/cm² or greater 35 nmoles/cm² or greater, 40 nmoles/cm² or greater, 45 nmoles/cm² or greater, 50 nmoles/cm² or greater, 55 nmoles/cm² or greater, 60 nmoles/cm² or greater, 65 nmoles/cm² or greater, 70 nmoles/cm² or greater, 75 nmoles/cm² or greater, 80 nmoles/cm² or greater, 85 nmoles/cm² or greater, 90 nmoles/cm² or greater, 95 nmoles/cm² or greater, 100 nmoles/cm² or greater, 200 nmoles/cm² or greater, 300 nmoles/cm² or greater, 400 nmoles/cm² or greater, 500 nmoles/cm² or greater, 1,000 nmoles/cm² or greater, 1,500 nmoles/cm² or greater, 2,000 nmoles/cm² or greater, 2,500 nmoles/cm² or greater, 3,000 nmoles/cm² or greater, from 1-3,000 nmoles/cm², from 1-1,500 nmoles/cm², from 1-500 nmoles/cm², from 10-100 nmoles/cm², from 10-200 nmoles/cm², from 10-500 nmoles/cm², from 15-80 nmoles/cm², from 30-100 nmoles/cm², or from 30-90 nmoles/cm², from 30-200 nmoles/cm², or from 30-500 nmoles/cm², and any value in between, e.g., 452 nmoles/cm²). In some embodiments, the density of the functional surface group (and the affinity reagent) is 3,000 nmoles/cm² or less (for e.g., 2,000 nmoles/cm² or less, 1,500 nmoles/cm² or less, 1,000 nmoles/cm² or less, 900 nmoles/cm² or less, 800 nmoles/cm² or less, 600 nmoles/cm² or less, 500 nmoles/cm² or less, 400 nmoles/cm² or less, 300 nmoles/cm² or less, 200 nmoles/cm² or less, or 100 nmoles/cm² or less, and any value in between, e.g., 1,278 nmoles/cm²).

Density control of the functional surface group (and the affinity reagent) can be achieved by a change in concentration of the reagent used to functionalize the solid surface. For example, in liquid phase, the concentration of the reagent (such as a silane or alkyne-containing reagent) can be varied to provide the desired density. In vapor phase methods such as CVD, the amount of silane and the deposition conditions (temperature, pressure, and duration of exposure) can be varied to provide the desired density. Alternatively, the density of the solid support is controlled by co-deposition of a second compound, on the surface. For example, a second silane group is deposited concurrently or sequentially with a first silane on a solid support. The relative density of the first functional surface group and the second functional surface group may vary depending on the characteristics of the functional group, solution conditions, and the like. A well-controlled density of the first and second functional surface group and/or affinity reagent on the surface of the solid support provides high nucleic acid binding capacity for the nucleic acid.

In one embodiment, the invention provides a solid support comprising a first functional surface group which is an alkylsilane and a second functional surface group which is an azidosilane. For example, the alkylsilane is deposited on the solid support first, followed by deposition of the azidosilane. Alternatively, the azidosilane is deposited on the solid support first, followed by deposition of the alkylsilane. In yet another alternative, the alkylsilane and azidosilane are deposited concurrently. The deposition may be performed, for example, by CVD. In some embodiments, the ratio of the first functional surface group and the second functional surface group can be less than 1:1, 1:2, 1:3, 1:5, or 1:10. In some embodiments, the ratio of the first functional surface group and the second functional surface group can be greater than 1:1, 1:2, 1:3, 1:5, or 1:10. In some embodiments, the ratio of the first functional surface group and the second functional surface group can be between 1:10 and 10:1, or between 1:10 and 5:1, or between 1:5 and 5:1, or between 1:5 and 1:2, or between 1:5 and 1:1.

The solid support together with the affinity reagent can have a DNA binding capacity of at least 10 μg/cm² (for e.g., 15 μg/cm² or greater, 20 μg/cm² or greater, 25 μg/cm² or greater, 35 μg/cm² or greater, 40 μg/cm² or greater, 45 μg/cm² or greater, 50 μg/cm² or greater, 55 μg/cm² or greater, 60 μg/cm² or greater, 65 μg/cm² or greater, 70 μg/cm² or greater, 75 μg/cm² or greater, 80 μg/cm² or greater, 85 μg/cm² or greater, 90 μg/cm² or greater, 95 μg/cm² or greater, 100 μg/cm² or greater, from 30-100 μg/cm² or from 30-90 μg/cm², or any value in between, e.g., 79 μg/cm²). In some embodiments, the solid support has a DNA binding capacity of at least 30 μg/cm².

### Affinity Reagent

As described herein, the methods utilize an affinity reagent capable of interacting with both the solid support and nucleic acid from the sample. Turning back to FIG. 1, the affinity reagent 20 comprises a first moiety 22 that interacts with and/or reacts with the solid support 10 and a second moiety 24 that interacts with and/or reacts with the nucleic acid 32. The affinity reagent may directly interact with and/or react with a surface of the solid support or indirectly interact with and/or react with the solid support via a functional surface group on the solid support. For example, the affinity reagent can be covalently or non-covalently (e.g., via ionic bonding, hydrophobic or van der Waals interactions, polar interactions) bonded directly to a surface the solid support. In other examples, the affinity reagent can be covalently or non-covalently bonded indirectly to the solid support via a functional surface group on a surface of the solid support. Both direct and indirect interactions and/or reactions are considered when referring to the affinity reagent interaction with and/or reaction with the solid support.

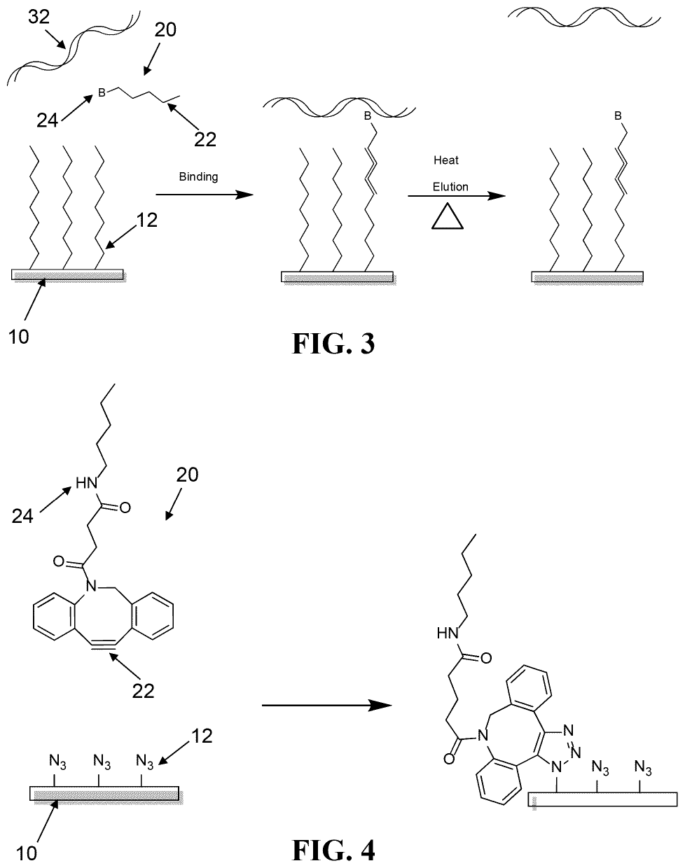

FIG. 2 shows an affinity reagent 20 that also comprises a photocleavable group 26, represented as X-X′. Once the nucleic acid is extracted and isolated from the sample, it is released from the solid support by photocleavage in the presence of an elution reagent. In some embodiments, the nucleic acid is extracted and isolated from the sample by releasing it from the solid support by heat, as shown in FIG. 3. FIG. 3 illustrates a solid support 10 modified with hydrophobic surface groups 12 and an affinity reagent 20 having a first moiety 22 that binds to the hydrophobic surface group 12 and a second moiety 24 that binds to the nucleic acid 32.

As described herein, the affinity reagent comprises a first moiety that interacts with and/or reactive with the solid support. The first moiety of the affinity reagent can comprise a functional group such as a silanizing group, an ether, a silyl ether, a siloxane, an ester of carboxylic acid, an ester of sulfonic acid, an esters of sulfamic acid, an ester of sulfuric acid, an ester of phosphonic acid, an ester of phosphinic acid, an ester of phosphoric acid, a silyl ester of carboxylic acid, a silyl ester of sulfonic acid, a silyl ester of sulfinic acid, a silyl ester of sulfuric acid, a silyl ester of phosphonic acid, a silyl ester of phosphinic acid, a silyl ester of phosphoric acid, an oxides, a sulfide, a carbocycle, a heterocycle with at least one oxygen atom, a heterocycle with at least one nitrogen atom, a heterocycle with at least one sulfur atom, a heterocycle with at least one silicon atom, a carbodiimide (such as DCC and EDCI), a phosphonium or imonium (such as BOP, PyBOP, PyBrOP, TBTU, HBTU, HATU, COMU, and TFFH), a ‘click’ reaction-derived heterocycle, a Diels-Alder reaction-derived carbocycle, a Diels-Alder reaction-derived heterocycle, an amide, an imide, a sulfide, a thiolate, a metal thiolate, a urethane, an oxime, a hydrazide, a hydrazone, a maleimide, an acrylate, an acrylamide, an epoxide, an aziridine, a thiirane, an aldehyde, a ketone, an azide, an alkyne, a disulfide, an anhydride, a carboxylates phosphate, a phosphonate, a sulfate, a sulfonate, a nitrate, an amidine, a silane, a siloxane (e.g., a cyclic siloxane), a triazinyl, a silatrane, a silsesquioxane, a silazane, a cyanate, an acetylene, a cyanide, a halogen, an acetal, a ketal, an amino, carbonyl, a carboxyl, biotin, cyclodextrin, an adamantane, a physisorbed or chemisorbed or otherwise non-covalently attached moiety, or a combination thereof. In some embodiments, the first moiety of the affinity reagent can comprise a functional group such as a hydrophobic group, a negatively charged binding group (e.g., sulfonic, sulfate, phosphoric, phosphonic or carboxylic group), a positively charged binding group (e.g., primary, secondary, tertiary amine and quaternary ammonium, heterocyclic amines, such as pyridine, pyrimidine, pyridinium, piperazine), a polar group (e.g., chemical moieties comprising polarized chemical bonds, such C—O, C═O, C—N, C═N, C═N, N—H, O—H, C—F, C—Cl, C—Br, C—S, S—H, S—O, S═O, C—P, P—O, P═O, P—H, more specifically carboxyl, alcohol, thiol, amide, halide, amine, ester, ether, or thioester), or a combination thereof, that interacts with the solid support. Hydrophobic groups, negatively charged binding groups, positively charged binding groups, and polar groups are as described herein. Preferably, the first moiety of the affinity reagent comprises a functional group that interacts with and/or reactive with the functional surface group on the solid support. For example, the interaction between the affinity reagent and the solid support may be via hydrophobic interactions. In that case, the affinity reagent comprises a first hydrophobic group, which interacts with the solid support comprising a second hydrophobic group. In some embodiments, the first and second hydrophobic groups are independently alkyl groups.

In some examples, the interaction with the solid support and the affinity reagent is based on fluorous affinity. In such cases, the affinity reagent and the solid support each comprises at least one fluorine atom substitution. For example, the affinity reagent and/or the solid support each independently comprises a perfluoroalkyl group.

In some examples, the interaction with the solid support and the affinity reagent is via a triazinyl moiety. Azide groups react rapidly and quantitatively with strained cyclic alkynes, generating a stable cycloadduct. Moreover, such chemical groups have minimal reactivity towards oligonucleotide functional groups (primary amines, secondary amines, primary alcohols, secondary alcohols, phosphates, ketones, aldehydes, etc.). A strained cyclic-alkyne and an azide can be used to create bio-orthogonal reactions for the functionalization of the solid support and affinity reagent. Accordingly, the use of azide and/or strained cyclic alkynyl modifications on the solid support or affinity reagent can serve as an appropriate way of functionalizing the solid support. In some examples, the interaction with the solid support and the affinity reagent is via an imidazole or indole moiety.

The affinity reagent further comprises a second moiety that interacts with and/or reactive with the nucleic acid in the sample. The second moiety can be described herein as a “DNA binding ligand”, a “nucleic acid binding ligand” or “nucleic acid binding moiety”. Nucleic acids will in general be any oligonucleotide and can include RNA or DNA, double stranded or single stranded, or having secondary (single stranded DNA or RNA can form internal double stranded regions, i.e., secondary structures) or tertiary structures.

The nucleic acid binding moiety includes any molecule which is capable of binding or associating with a nucleic acid. This binding or association may be via covalent bonding, via ionic bonding, via hydrogen bonding, via Van-der-Waals bonding, or via any other type of reversible or irreversible association. The term “moiety” is used herein to refer to any atom, ion, molecule, macromolecule (for example polypeptide), or combination of such entities. The term “binding moiety” is used interchangeably with the term “ligand”.

The nucleic acid binding moiety can be selected from a primary amine, a secondary amine, a triazole, an imidazole, an indole containing compound (such as an alkylamine, a cycloalkylamine, an alkyloxy amine, a polyamine, or an arylamine) or other heterocyclic moieties (such as aziridine, azirine, pyrrolidine, pyrroline, pyrrole, pyrazolidine, imidazoline, pyrazole, tetrazole, oxazole, thiadiazole, piperidine, piperazine, pyrimidine, pyrazine, triazine, oxazine, morpholine, isoindole, indazole, sazaindole, azaindazole, purine, adenine, guanine, quinoline, quinozaline, cinnoline, pteridine), an intercalating agent (e.g., furocoumarins, coumarins, anthracyclines, phenanthridines, psoralen derivatives, acridines, ellipticines, actinomycins, anthracenediones, and Tris compounds), a groove binder (e.g., pyrrolo(1,4)benzodiazepines (PBD's), anthelvencins, kikumycins, netropsin, distamycin, calicheamicin, CC-1065, and Hoechst 33258), a polypeptide, an amino acid (histidine), a protein (such as zinc finger proteins, homeodomains, leucine zipper proteins, helix-loop-helix proteins or β-sheet motifs), or a combination thereof.

In some aspects of the affinity reagent disclosed herein, the nucleic acid binding moiety can comprise a plurality of amine groups (or a polyamine), a plurality of amide groups (or a polyamide), or a combination thereof (a polyamine-amide). For example, the nucleic acid binding moiety can comprise at least two, at least three, at least four, at least five, at least six amine or amide groups, or a combination thereof. In other examples, the nucleic acid binding moiety comprises a single amine or amide group. The amine or amine group can be a primary, secondary, or tertiary amine. In some embodiments, the nucleic acid binding moiety comprises a trialkylammonium group such as triethylammonium acetate or quaternary ammonium group.

In some aspects of the affinity reagent, the nucleic acid binding moiety comprises a C₁-C₁₆ alkylamine (e.g., C₁-C₁₂ alkylamine, C₁-C₁₀ alkylamine, C₁-C₈ alkylamine, C₂-C₈ alkylamine, or C₂-C₆ alkylamine), a C₃-C₁₂ cycloalkylamine (e.g., C₃-C₁₀ cycloalkylamine, C₃-C₈ cycloalkylamine, C₃-C₆ cycloalkylamine, C₄-C₈ cycloalkylamine, or C₄-C₆ cycloalkylamine), an C₁-C₁₆ alkyloxy amine (e.g., C₁-C₁₂ alkyloxy amine, C₁-C₁₀ alkyloxy amine, C₁-C₈alkyloxy amine, C₂-C₈ alkyloxy amine, or C₂-C₆ alkyloxy amine), a C₆-C₁₂ arylamine (e.g., C₆-C₁₀ arylamine, C₆-C₈ arylamine), a C₆-C₁₂ imidazole group, a C₃-C₁₄ hetero cycloalkylamine (e.g., C₃-C₁₀ hetero cycloalkylamine, C₃-C₈ hetero cycloalkylamine, C₃-C₆ hetero cycloalkylamine, C₄-C₈ hetero cycloalkylamine, or C₄-C₆ hetero cycloalkylamine), and a C₂-C₂₀ heteroalkylamine (e.g., C₁-C₁₂ heteroalkylamine, C₁-C₁₀ heteroalkylamine, C₁-C₈ heteroalkylamine, C₂-C₈ heteroalkylamine, or C₂-C₆ heteroalkylamine), or a combination thereof. In some embodiments, the nucleic acid binding moiety comprises an alkylamine group, an imidazole group, or a combination thereof.

In some embodiments, the affinity reagent comprises a nucleic acid binding moiety which interacts with the nucleic acid by an electrostatic interaction. Such nucleic acid binding moieties may be amines or other nitrogen-containing groups which can bind a nucleic acid with higher affinity when positively charged (at a relatively lower pH) and release the nucleic acid when neutral (at a relatively higher pH). In some examples, the nucleic acid binding moiety is an imidazole. In other examples, the nucleic acid binding moiety is selected from spermidine, spermine, methylamine, ethylamine, propylamine, cadaverine, putrescine, ethylenediamine, diethylene triamine, 1,3-dimethyldipropylenediamine, 3-(2-aminoethyl)aminopropyl, (2-aminoethyl)trimethylammonium hydrochloride, tris(2-aminoethyl)amine, or a combination thereof.

The nucleic acid binding moiety can be an intercalator or a groove binder (major or minor groove). Intercalators include, but are not limited to, daunomycin, ditercalinium, dactinomycin, adriamycine, and pyrene. Minor groove binding moieties include for example a polyamide such as a heterocyclic polyamide (comprising e.g. pyrrole and/or imidazole groups). Other minor groove binding moieties include small molecules such as netropsin, distamycin, pentamidine, berenil, and doxorubicin.

The nucleic acid binding moiety can be a fluorescent dye. Suitable fluorescent dyes include PicoGreen, OliGreen, EvaGreen, GelGreen, GelRed, ethidium bromide, propidium iodide, Acridine orange, 7-aminoactinomycin D, cyanine dyes, bisbenzimide, benzoxanthene yellow, netropsin, crystal violet, any SYTO dye, SYBR Green I, SYBR Green II, SYBR DX, OliGreen, CyQuant GR, SYTOX, POPO-1, BOBO-1, YOYO-1, TOTO-1, JOJO-1, POPO-3, LOLO-1, BOBO-3, YOYO-3, TOTO-3, PO-PRO-1, YO-PRO-1, TO-PRO-1, JO-PRO-1, PO-PRO-3, YO-PRO-3, TO-PRO-3, TO-PRO-5, DAPI, Hoechst 33258.

The first moiety (that binds to the solid support) and second moiety (nucleic acid binding moiety) of the affinity reagent can be conjugated via a linker. In some embodiments, the linker is a branched group conjugated to the first moiety or the second moiety. The linker can be selected from an alkyleneoxy (e.g., a C₂-C₄ alkyleneoxy) group, an alkylene (e.g., a C₂-C₄ alkylene or C₂-C₃ alkylene) group, or a heteroalkylene (e.g., C₄-C₆ heteroalkylene). In some examples, the linker is a bond. In other examples, the linker comprises a polyethylene glycol, an alkyl group, an ester, amide, disulfide, carbamate, or thiol group. In further examples, the linker can comprise a photocleavable group, that allows convenient release of the nucleic acid from the solid support. Suitable photocleavable groups include nitrobenzyl linkers, coumarin linkers, anthracene linkers, and quinone linkers.

In some embodiments, the linker comprises one or more functional groups that interact with a solvent in the sample to improve solubility of the affinity reagent. For instance, when the linker comprises polyethylene glycol (PEG) or a similar polymer group, the linker changes the physical and chemical properties of the affinity reagent, such as its conformation, electrostatic binding, and hydrophobicity. In some examples, the affinity reagent with one or more PEG linkers changes the solubility of the affinity reagent in water or other solvents.

In some embodiments, the linker comprises ester-based linkers (such as branched ester-based linkers), amide-based linkers (such as branched amide-based linkers including N,N-dihexyl-3-imidazole-2-oxoamine linkers), amine-based linkers (such as 3-dibutylaminopropylamine linkers, N,N-dihexyl-3-aminopropylamine linkers), organophosphorous-based linkers (such as organophosphine-based linkers, organophosphine oxide-based linkers, organophosphinate-based linkers, organophosphoramidate-based linkers, organophosphate-based linkers, organophosphonamidate-based linkers, or organophosphonate-based linkers), glucuronic acid-based linkers, disulfide linkers, cathepsin B linkers, or combinations thereof.

In some examples, the affinity reagent comprising the first and second moieties is an alkylamine. The alkyl group can be linear or branched. In some embodiments, the alkylamine is methylamine, ethylamine, diethylamine, triethylamine, propylamine, isopropylamine, diisopropylamine, butylamine, isobutylamine, sec-butylamine, tert-butylamine, pentylamine, isopentylamine, hexylamine, isohexylamine, cyclohexylamine, heptylamine, octylamine, nonylamine, decylamine, undecylamine, dodecylamine, tridecylamine, tetradecylamine, pentadecylamine, hexadecylamine, heptadecylamine, octadecylamine, nonadecylamine, or 3-dibutylamino propylamine.

### Cartridges

In some embodiments, the solid support and the affinity reagent are incorporated into an automated cartridge, such as a GeneXpert® cartridge. In one aspect, the invention pertains to a sample cartridge that utilizes a valve body platform that allows for detection of enveloped and free nucleic acid targets. In some embodiments, the valve body includes a sample processing region or lysing chamber that provides for either or both mechanical and chemical lysis. This allows a single cartridge to provide lysing for a multitude of differing types of targets, thus, can be considered an “assay panel cartridge.”

The sample cartridge can be any device configured to perform one or more process steps relating to preparation and/or analysis of a biological fluid sample according to any of the methods described herein. In some embodiments, the sample cartridge is configured to perform at least sample preparation. The sample cartridge can further be configured to perform additional processes, such as detection of a target nucleic acid in a nucleic acid amplification test (NAAT), e.g., Polymerase Chain Reaction (PCR) assay, by use of a reaction vessel attached to the cartridge. In some embodiments, the reaction vessel extends from the body of the sample cartridge. Preparation of a fluid sample generally involves a series of processing steps, which can include chemical, electrical, mechanical, thermal, optical or acoustical processing steps according to a specific protocol. Such steps can be used to perform various sample preparation functions, such as cell capture, cell lysis, binding of analyte, and binding of unwanted material.

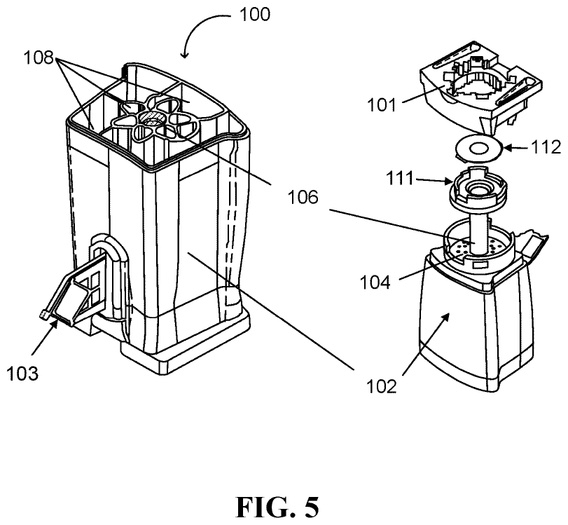

A cartridge suitable for use with the invention, includes one or more transfer ports through which the prepared fluid sample can be transported into an attached reaction vessel for analysis. FIG. 5 illustrates an exemplary assay panel cartridge 100 suitable for sample preparation and analytics testing by PCR when received in an instrument module in accordance with some embodiments. The sample cartridge is configured for performing a variety of sample processes, including chemical lysing of targets, which is configured for immuno-PCR and optional integrated nucleic acid analysis of the target assay panel in accordance with some embodiments of the invention.

The sample cartridge is attached with a reaction vessel 103 (also referred to as a “reaction tube” or “PCR tube”) adapted for analysis of a fluid sample processed within the sample cartridge 100. In some embodiments the reaction vessel extends from the cartridge body. Such a sample cartridge 100 includes various components including a main housing 102 having one or more chambers 108 for processing of the fluid sample, which typically include sample preparation before analysis. The instrument module facilitates the processing steps needed to perform sample preparation and the prepared sample is transported through one of a pair of transfer ports into fluid conduit of the reaction vessel 103 attached to the housing of the sample cartridge 100. The prepared biological fluid sample is then transported into a reaction chamber of the reaction vessel where the biological fluid sample undergoes nucleic acid amplification. In some embodiments, the amplification is a polymerase chain reaction. In some embodiments, concurrent with the amplification of the biological fluid sample, an excitation means, and an optical detection means of the module is used to detect optical emissions that indicate the presence or absence of a target nucleic acid analyte of interest, e.g., a bacteria, a virus, a pathogen, a toxin, a tumor, or other target analyte. It is appreciated that such a reaction vessel could include various differing chambers, conduits, or micro-well arrays for use in detecting the target analyte. The cartridge is provided with means to perform preparation of the biological fluid sample before transport into the reaction vessel. Any chemical reagent required for cell lysis, or means for binding or detecting an analyte of interest (e.g., reagent beads) can be contained within one or more chambers of the cartridge, and as such can be used for sample preparation.

An exemplary use of a reaction vessel for analyzing a biological fluid sample is described in commonly assigned U.S. Pat. No. 6,818,185, entitled “Cartridge for Conducting a Chemical Reaction,” filed May 30, 2000, the entire contents of which are incorporated herein by reference for all purposes. Examples of the sample cartridge and associated modules are shown and described in U.S. Pat. No. 6,374,684, entitled “Fluid Control and Processing System” filed Aug. 25, 2000, and U.S. Pat. No. 8,048,386, entitled “Fluid Processing and Control,” filed Feb. 25, 2002, U.S. Patent Application No. 63/218,672 entitled “Universal Assay Cartridge and Methods of Use” filed Jul. 1, 2021; U.S. Provisional Application No. 63/319,993 entitled “Unitary Cartridge Body and Associated Components and Methods of Manufacture” filed Mar. 15, 2022; and U.S. Pat. No. 10,562,030 entitled “Molecular Diagnostic Assay System” filed Jul. 22, 2016; the entire contents of which are incorporated herein by reference in their entirety for all purposes.

In some examples, the reaction vessel comprises integrated optical sensor circuits. The integrated optical sensor circuit may comprise a CMOS substrate. A CMOS substrate may be the solid and planar surface of a semiconductor device that is fabricated using complementary metal-oxide-semiconductor microfabrication processes. Such a device may have dimensions (width and length) between 0.5 mm to 25 mm and a thickness of between 0.05 mm to 1.5 mm. The surface of CMOS substrates may be microfabricated using planar very large-scale integration (VLSI) processes to have electrically non-conductive materials, generally referred to as the passivation layer. In some cases, the passivation layer on the CMOS substrate is optically transparent to allow the CMOS device to perform optical detection (e.g., of a fluorescent signal). Example materials for the passivation layer are thin oxides such as SiO₂ and Si₃N₄.

The compositions, methods, and systems described herein may be part of an optical biochip system. An optical biochip system may comprise a CMOS biosensor array that uses optical detection methods, such as inverse fluorescence transduction (IFT) (see, e.g., U.S. Pat. No. 9,133,504 and Hassibi, A et al. Nat. Biotechnol. 2018, 36 (8), 738-745). Such optical biochip systems may be part of systems or used in methods for molecular detection assays in life-science research and diagnostics, such as applications using nucleic acid molecules s their detection targets or molecular recognition elements (e.g., probe). Some examples of such applications are nucleic acid amplification tests that use polymerase chain reaction processes, affinity-based detection systems that take advantage of 2-dimensional and addressable DNA microarrays, and DNA sequencing arrays that incorporate solid-phase sequencing by synthesis (SBS) methods.

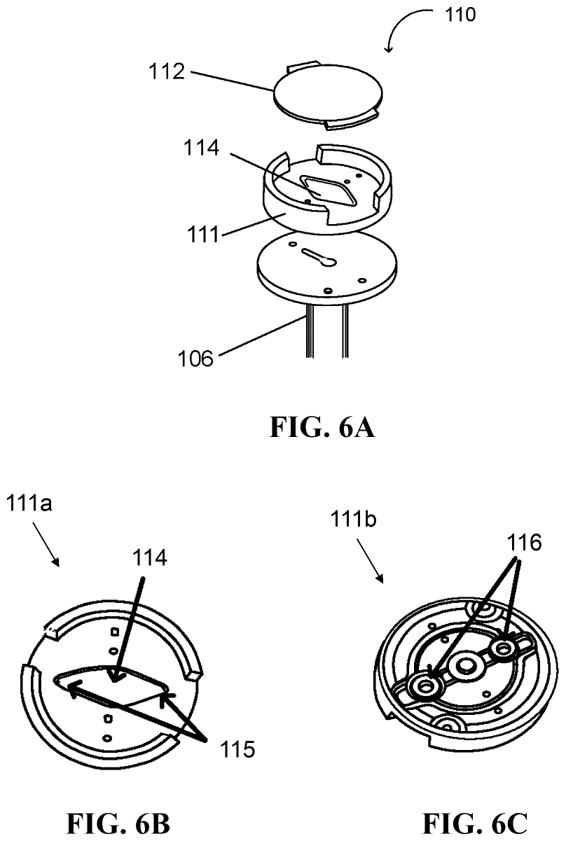

As shown in FIG. 5, the assay panel cartridge 100 comprises a cartridge body 102 containing a plurality of chambers 108 for reagents or buffers and sample processing. The chambers are disposed around a central syringe barrel 106 that is in fluid communication with a valve assembly 110 comprising a valve body 111 and that is sealed with a gasket 104. The valve body 111 can include a cap 112 and the entire cartridge body can be supported on a cartridge base 101. As shown in more detail in FIGS. 6A through 6C, the valve body 111 typically contains one or more channels or cavities (chamber(s) 114) that can contain ports 115 on a front surface 111a of the valve body (FIG. 6B) and screens 116 on a back surface 111b of the valve body (FIG. 6C) of the valve body.

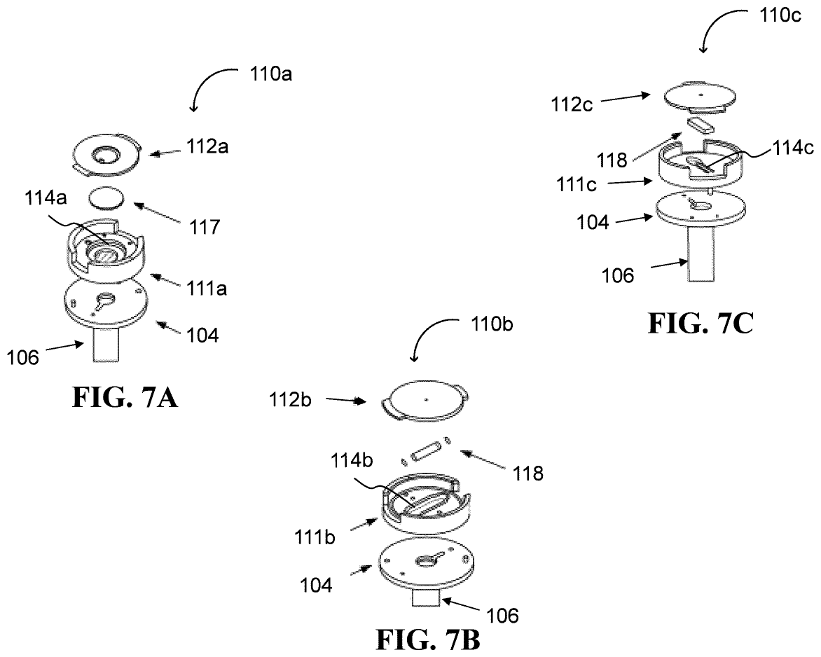

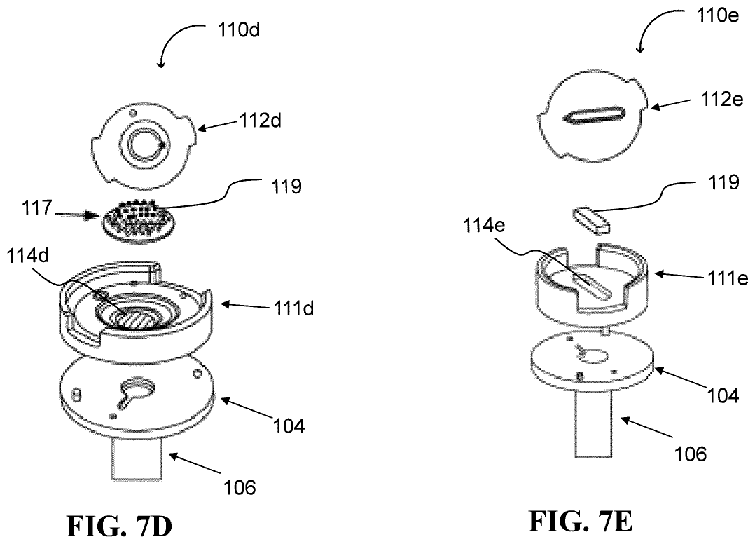

FIGS. 7A through 7E demonstrate various examples of valve assemblies 110a-110c within the assay panel cartridge 100. For instance, the valve assembly of FIG. 7A contains a valve body 111a, a cap 112a, a filter membrane 117, a gasket 104, and a central syringe barrel 106. The valve body 111a contains a channel 114a that supports the filter membrane 116. FIGS. 7B-7E illustrate how valve assembly 110b-110e can be modified to accommodate other shapes, sizes, and orientations of valve bodies 111b-111e containing a channel 114b and a filter membrane 117 and filter column 118. Filter membrane 117 and filter column 118 can be a suitable separating material described herein that can function to bind and elute a nucleic acid. In some embodiments, as shown in FIG. 7D, filter membrane 117 can include filter particles or beads 119. Filter particles or beads 119 have greater surface area than filter membrane 117 and can be designed to bind and elute high concentrations of nucleic acid.

The valve assemblies of FIGS. 7A-7C demonstrate how each cartridge can be suited for one or more types of target lysing, any of which may be used in a respective sample cartridge. Each cartridge includes a filter material having a surface and reagent chemistry, in accordance with embodiments described herein. A cartridge with a valve assembly shown in FIG. 7A performs mechanical lysing for more hardy targets and includes a filter membrane modified with a DNA binding ligand. Cartridges with the valve assemblies shown in FIGS. 7B and 7C perform chemical lysing for viruses, free NA or more fragile targets and include a filter column. In some embodiments, a valve assembly includes a modified filter shown in FIGS. 7D and 7E, preferably with glass particles or a modified glass fiber filter 119 and is sized to be secure between the valve cap and valve body. The modified glass fiber filter 119 utilizes glass beads on the modified glass fiber filter suited for mechanical lysis of certain types of targets. In this embodiment, the modified filter 119 is formed of glass fibers and has a 0.7 μm pore size. In contrast, the filter in FIG. 7D utilizes a filter formed as a disk of a polymer film (i.e., PCTE), which while suitable for mechanical lysing, but not suited for chemical lysing. By utilizing a filter having a pore size of 0.7 μm, the filter is suitable for receiving suitably sized glass beads for mechanical lysing. Utilizing glass fibers to form the filter facilitates affinity bonding with the free nucleic acid released by chemical lysing. Thus, this filter is suited for both mechanical and chemical lysing.

In some embodiments the cartridge further comprises one or more temperature-controlled channels or chambers that can, in certain embodiments, function as thermocycling chambers. A “plunger” not shown can be operated to draw fluid into the syringe barrel 106 and rotation of the valve body 111 provides selective fluid communication between the various reagent chambers 108 and channels, reaction chamber(s), mixing chambers, and optionally, any temperature-controlled regions. Thus, the various reagent chambers 108, reaction chambers, matrix material(s), and temperature-controlled chambers or channels are selectively in fluid communication by rotation of the plunger and reagent movement (e.g., chamber loading or unloading) is operated by the “syringe” action of the plunger within the valve assembly.

Turning back to FIGS. 7A through 7E, different valve assembles 110 can be used in the sample cartridge of FIG. 5. For instance, the valve assembly of FIG. 7A is configured to perform mechanical lysing and is suitable for lysing hardy targets (e.g., certain bacteria, spores). The valve assemblies shown in FIGS. 7B and 7C are configured to perform chemical lysing and are suitable for lysing less hardy targets (e.g., viruses, free NA, some spores, some bacteria and yeasts). The universal valve assembly can perform both mechanical and chemical lysing for all types of targets. In all such cartridges, the valve assembly includes the syringe tube 106, valve body 111a-111c, and valve cap 112a-112c. The capabilities of the valve assembly of the sample cartridge rely on the filter or separating material described herein, as well as the particular workflow sequence performed by the instrument interface of the module. For example, valve assembly 110a (FIG. 7A) has a valve body shaped with a circular cavity to support a filter disc to filter the sample, and the cap has a sonication dome feature, which interfaces with a sonication horn of a cartridge receiving module so as to ultrasonically lyse the target. By contrast, valve assemblies 110b and 110c (FIGS. 7B and 7C) have a valve body with an oblong filter recess that receives a filter (e.g., glass fiber filter column) therein, the filter configured for binding with nucleic acid released from the target optionally by chemical lysing. The universal valve assembly has a design more similar to valve assembly 110a of FIG. 7A, having a cap with a sonication dome, and a valve body with a circular cavity for supporting a disc filter, however this design uses a glass fiber filter as described herein. Utilizing the DNA binding ligands with the filter material (such as modified glass fibers) to form the filter facilitates affinity bonding with the free nucleic acid released from the biological sample, optionally by chemical lysing.

While the compositions and methods described herein are described primarily with reference to the GENEXPERT® cartridge by Cepheid Inc. (Sunnyvale, Calif.) (see, e.g., FIG. 5), it will be recognized, that in view of the teachings provided herein the methods can be implemented on other cartridge/microfluidic systems, including alternative cartridge designs having valve assemblies that involve multiple interfacing components, as well as cartridge body defined by multiple interfacing components to form the multiple chambers of the cartridges, for example, those described in Korean Application No. 102293717B1, cartridges that utilizes ultrasonic waves to lyse cells in a biological sample, for example, those described in International Application No. WO2021/245390A1, cartridges and systems that utilizes an electrowetting grid for microdroplet manipulation and electrosensor arrays configured to detect analytes of interest, for example, those described in International Application No. WO2016/077341A2, cartridges that facilitate movement of nucleic acid from one chamber to the next chamber by opening a vent pocket, for example, those described in International Application No. WO2012/145730A2, assay systems comprising an addressable array of nucleic acid probes attached to a solid surface, for example, those described in U.S. Pat. No. 9,708,647, multiplexed assay systems comprising a plurality of thermocycling units such that individual chambers can be heated, cooled, and/or compressed to mix fluid within the chamber or to propel fluid in the chamber into another chamber, for example, those described in International Application No. WO2015/138343A1, and as well as systems for rapid amplification of nucleic acids facilitated by flexible portions of the sample cartridge aligned to accomplish temperature cycling for nucleic acid amplification, for example, those described in International Application No. WO2017/147085A1. Additionally, it is appreciated that the methods described herein can further be realized in entirely different systems, including: isothermal nucleic acid amplification systems, Digital RT-PCR, Electrochemical PCR, lateral flow testing cartridges, electrochemical sensors, nucleic acid sequencing, CRISPR/Cas BASED technologies, chemiluminescence, and nanoparticle-based colorimetric detection.

In some embodiments, the sample cartridge can comprise a) a cartridge body having a plurality of chambers defined therein, wherein the plurality of chambers are in fluidic communication through a fluidic path of the cartridge, and wherein at least one chamber is configured to receive the biological sample, b) a reaction vessel configured for amplification of the nucleic acid by thermal cycling, and c) a filter disposed in the fluidic path between the plurality of chambers and the reaction vessel, wherein the filter comprises a solid support material as disclosed herein, and wherein the plurality of chambers and the reaction vessel independently comprise an affinity reagent and other reagents for releasing nucleic acid from a biological sample, and primers and probes for detection of the nucleic acid.

In other embodiments, the sample cartridge can comprise a) a cartridge body having a plurality of chambers therein, wherein the plurality of chambers includes a sample chamber configured to receive the biological sample; b) a reaction vessel fluidically coupled to the plurality of chambers and configured for amplification of nucleic acid and detection of a plurality of amplification products; c) a filter disposed in the fluidic path between the plurality of chambers and the reaction vessel, wherein the filter comprises a solid support having a surface capable of binding an affinity reagent; d) the affinity reagent disposed in one of the plurality of chambers, the affinity reagent comprising a first moiety that interacts with the filter and a second moiety that interacts with nucleic acid from the biological sample; and c) primers and/or probes disposed in one or more chambers of the plurality of chambers or reaction vessel for detection of the nucleic acid. In some embodiments, the sample cartridge is a biosensor cartridge, such as those described in U.S. Pat. No. 11,833,503B2, wherein the primers and/or probes are immobilized on a surface (e.g., a biosensor surface) in one or more chambers of the plurality of chambers or reaction vessel for detection of the nucleic acid. The solid support and affinity reagent used in the sample cartridge are as described herein.

The lysis chamber optionally comprises lysis reagents, the lysis reagents selected from a chaotropic agent, a chelating agent, a buffer, and a detergent. The lysis chamber may further comprise a valve body and a valve cap, wherein the valve body interfaces with the valve cap to define the lysis chamber therebetween, and wherein the filter is held within the lysis chamber secured between the valve body and the valve cap.

The lysis chamber has a fluid flow path between an inlet in the cap and an outlet in the valve body that is fluidically coupled to a fluid displacement region of the valve body, wherein the fluid displacement region is depressurizable by movement of the syringe to draw fluid into the fluid displacement region and pressurizable by movement of the syringe to expel fluid from the fluid displacement region. The sample cartridge together with the reagents can allow for flow rates up to about 100 μL per second, such as from about 10 μL to about 100 μL. The sample cartridge together with the reagents can allow for pressure below 100 psi, below 80 psi, or below 60 psi. The sample cartridge can allow for sample volumes up to 1000 μL, such as from 300 μL to 1,000 μL.

The cartridge body can further comprise an ultrasonic, piezoelectric, magnetostrictive, or electrostatic transducer, for example in the lysis chamber to facilitate mechanical lysing. The sample cartridge may further comprise a syringe that is movable to facilitate fluid flow into and from the lysis chamber by fluctuation of pressure.

The cartridge can be a single-use disposable cartridge. In some embodiments, the cartridge is an automated cartridge.

In order to increase sensitivity of detection, large sample volumes can be prepared. As described herein, the preparation of large volumes, however, is contradictory to microfluidic systems for automatic lysis, processing and/or analysis of biological samples. There is therefore a demand for solutions which permit preparing a large sample volume by means of, for example, filtration, and to make the isolated nucleic acids available in a small volume to a microfluidic system via a microfluidic interface. The sample cartridges comprising a filter having a surface capable of binding to the affinity reagent and methods described herein are used for accomplishing this need. The cartridges and methods allow for the detection of target nucleic acid (e.g., DNA) from various sample types (including whole blood, plasma, serum, semen, spinal fluid, tissue, tear, urine, stool, saliva, respiratory sample, nasopharyngeal sample, vaginal swab, vaginal mucus sample, vaginal tissue sample, vaginal cell sample, bacterial culture, mammalian cell culture, viral culture, human cell, bacteria, extracellular fluid, pancreatic fluid, cell lysate, PCR reaction mixture, or in vitro nucleic acid modification reaction mixture) without requiring the user to take excessive sample processing steps.

The method for processing large volume samples includes introducing the biological sample into the sample cartridge. In some instances, the sample can be mixed with reagents prior to introducing it into the cartridge, to disrupt particulates present within the sample. However, the sample introduced into the sample cartridge may also be disrupted in the sample cartridge only when the processing is being carried out.

In order to be able to process a large volume, the volume of the sample chamber and/or lysis chamber within the sample cartridge can be in particular at least 300 μL, at least 500 μL, at least 1,000 μL, at least 1,500 μL, at least 2,000 μL, at least 2,500 μL, at least 3,000 μL, at least 3,500 μL, at least 4,000 μL, at least 4,500 μL, at least 5,000 μL, at least 5,500 μL, at least 6,000 μL, at least 6,500 μL, at least 7,000 μL, at least 7,500 μL, at least 8,000 μL, at least 8,500 μL, at least 9,000 μL, at least 9,500 μL, or at least 10,000 μL. Preferably, the sample cartridge can purify and process nucleic acid from a liquid sample up to 10,000 μL in volume, such as from 300 μL to 5,000 μL, from 300 μL to 3,000 μL, from 300 μL to 2,000 μL, or from 300 μL to 1,000 μL in volume.

It is possible in some cases to disrupt a biological sample in the sample cartridge with a lysis buffer, that is, a solution. In other cases, the biological sample is disrupted prior to introducing into the sample cartridge. Often, it is desirable to treat the sample with enzymes such as lysozyme, proteinase K, and/or a leukoreduction agent before mixing the sample with a chemical lysis buffer. Also, it may be desirable to have more than one lysis buffer and more than one wash buffer. Similarly, aspects of the instrument are not shown that may be used to improve the efficiency of extraction and purification of the large sample volume. For example: 1) a prefilter for capturing the particulate matter in the sample prior to or after lysis, but before sending the lysate over the modified nucleic acid binging filter; 2) elements involved in heating the sample during/prior to lysis, 3) elements involved in sonicating or shearing the sample during lysis, 4) elements involved in sending heated or de-humidified air over the nucleic acid binding matrix that improve drying, and similar features are not shown, but can be assumed to be included to improve the overall performance of the instrument. In some embodiments, multiple rounds of drawing the sample in, then directing the ‘filtered’ sample to waste, can be completed until all the sample or the sample container is left empty.

### Methods

### Isolation of Nucleic Acid

Also provided herein are methods for isolation and purification of a nucleic acid from a nucleic acid containing sample using the compositions disclosed herein. The nucleic-acid containing sample can be selected from blood, plasma, serum, semen, a vaginal swab, a vaginal mucus sample, a vaginal tissue sample, a vaginal cell sample, spinal fluid, tissue, tear, urine, stool, saliva, smear preparation, bacterial culture, mammalian cell culture, viral culture, human cell, bacteria, extracellular fluid, PCR reaction mixture, paraffin-embedded tissue sample, cell lysate, or in vitro nucleic acid modification reaction mixture. In specific embodiments, the nucleic-acid containing sample (biological sample) can be a fixed paraffin-embedded samples (e.g., from FFPET samples) which can be used to identify the presence and/or the expression level of a gene, and/or the mutational status of a gene. In some embodiments, the nucleic acid containing sample can be a liquid biopsy sample for detection of cancer such as prostate, lung, breast, pancreas, colon, esophagus, ovary, bile duct, stomach, and liver cancers. In some embodiments, the nucleic acid containing sample can be a respiratory sample for detection of an infectious disease. The nucleic acid-containing sample may comprise human, bacterial, fungal, animal, or plant material. In other embodiments, the nucleic acid-containing sample can be obtained from a nucleic acid modification reaction or a nucleic acid synthesis reaction.

Nucleic acid encompasses any synthetic or naturally occurring nucleic acid, such as DNA or RNA, in any possible configuration, i.e., in the form of double-stranded nucleic acid, single-stranded nucleic acid, aptamer, or any combination thereof. The nucleic acid can be DNA, including dsDNA, ssDNA, and their hybrids. The nucleic acid can also be RNA, such as an mRNA, a non-coding RNA, total RNA, and the like. The nucleic acid can be a synthetic nucleic acid. In some embodiments, the nucleic acid is isolated using the methods described herein are well suited for use in diagnostic methods, prognostic methods, methods of monitoring treatments (e.g., cancer treatment), and the like. Accordingly, the target nucleic acid can comprise genomic DNA, total RNA, short-DNA, small DNA, tumor-derived nucleic acid (including circulating tumor DNA), methylated DNA, microbial nucleic acid, bacterial nucleic acid, viral nucleic acid, cell free nucleic acid, or combinations thereof. In some embodiments, the nucleic acids isolated using the methods described herein are utilized to detect the presence, and/or copy number, and/or expression level, and/or mutational status of one or more cancer markers.

The method for isolation of a nucleic acid from a nucleic acid containing sample can comprise (a) contacting a solid support with the sample and an affinity reagent, wherein the affinity reagent comprises a first moiety that interacts with the solid support and a second moiety that interacts with the nucleic acid from the sample, and (b) concentrating the nucleic acid onto the solid support. In some embodiments, the sample is a biological sample which is contacted with a lysis solution prior to contacting with the solid support, thereby lysing the cells contained in the biological sample and releasing the nucleic acids into solution.

When used, the lysis solution may comprise a chaotropic agent, such as guanidinium thiocyanate, guanidinium hydrochloride, alkali perchlorate, alkali iodide, urea, formamide, and combinations thereof. In some embodiments, the lysis solution may comprise a salt, such as a sodium chloride or calcium chloride salt. In some examples, the lysis buffer comprises one or more of a chaotropic agent, a salt, a buffering agent, a surfactant, a defoaming agent, or a combination thereof. The sample can be lysed by contacting the sample with a lysis buffer prior to contacting the sample with the solid support and subsequent precipitation of nucleic acids. In some instances, the lysis solution comprises one or more proteases. Suitable proteases include, but are not limited to serine proteases, threonine proteases, cysteine proteases, aspartate proteases, metalloproteases, glutamic acid proteases, metalloproteases, and combinations thereof. Illustrative suitable proteases include, but are not limited to proteinase k (a broad-spectrum serine protease), subtilysin trypsin, chymotrypsin, pepsin, papain, and the like. Using the teaching and examples provided herein, other proteases will be available to one of skill in the art.

In some embodiments, the methods disclosed herein do not require the use of a chaotropic reagent or high salt concentration for lysing the nucleic acid containing sample. In some embodiments, the methods disclosed herein can require lower concentrations of a chaotropic reagent or salt for lysing the nucleic acid containing sample, compared to conventional lysis assays. For example, the chaotropic agent can be used in concentrations of less than 4.5 M, less than 2 M, or less than 1 M. In some embodiments, the methods disclosed herein do not require the use of a lysis buffer comprising chaotropic agent.

The method of isolating the nucleic acid can further comprise filtering, centrifuging, precipitating, and/or washing the nucleic acid to concentrate the nucleic acid, prior to elution. Conventionally, after nucleic acid lysis, the lysate is filtered on a solid support in the presence of a binding agent (such as PEG) to bind the nucleic acid to the solid support. The binding agent can comprise one or more of an alcohol (e.g., methanol, ethanol, propanol, isopropanol), an alkane diol or alkane triol having 2 to 6 carbon atoms, a monocarboxylic acid ester or dicarboxylic acid diester having 2 to 6 carbon atoms in the acidic component and 1 to 4 carbon atoms in the alcoholic component; a (poly)ethylene glycol and ether derivatives and ester derivatives thereof, and a poly(4-styrene sulfonic acid-co-maleic acid). For example, the binding agent can include one or more of 1,2-butanediol, 1,2-propanediol, 1,3-butanediol, 1-methoxy-2-propanol acetate, 3-methyl-1,3,5-pentanetriol, DBE-2, DBE-3, DBE-4, DBE-5, DBE-6, diethylene glycol monoethyl ether (DGME), triethylene glycol monoethyl ether (TGME), diethylene glycol monoethyl ether acetate (DGMEA), ethyl lactate, ethylene glycol, poly(2-ethyl-2-oxazoline), poly(4-styrene sulfonic acid-co-maleic acid) sodium salt solution, tetraethylene glycol (TEG), tetraglycol, tetrahydrofurfuryl polyethylene glycol 200, tri (ethylene glycol) divinyl ether, anhydrous triethylene glycol, and triethylene glycol monoethyl ether. In some embodiments of the methods disclosed herein, a binding agent is not required, or lower concentrations of binding agents can be used compared to conventional assays. For example, the binding agent such as PEG can be used in concentrations of less than 40% v/v, less than 30% v/v, less than 20% v/v, or less than 10% v/v, of the filtering agent and/or the washing agent. The solid support together with the affinity reagent described herein allow selective capture of nucleic acids (RNA and DNA) from biological matrices. Indeed, the solid support together with the affinity reagent can capture free circulating nucleic acid as well as nucleic acid from cells without the use of a lysis buffer, salt, or binding agent or with very low concentrations of the same. It is important to point out that the invention described herein encompasses capture of complex genomic DNA or RNA from various organisms in biological samples. Modified glass microscope slides, for example, are commonly used to immobilize DNA or RNA for microarray imaging. In general, these flat, modified surfaces have very low surface area and are not suitable for isolating DNA or RNA from large volumes of complex samples.

The concentrated nucleic acid can be optionally washed on the solid support for example, to remove components of the lysis buffer or unwanted components from the biological sample. The concentrated (bonded) nucleic acid can be washed in a buffer compatible with PCR reactions.

The nucleic acid is subsequently eluted from the solid support with an elution buffer. Elution of the nucleic acids off the solid support can be achieved by increasing the pH of the eluent mobile phase or eluting agent, stepwise or in a gradient manner. In some embodiments, the bonded nucleic acid can be eluted from the solid support by contacting with an alkali solution. The alkali solution can comprise ammonia or an alkali metal hydroxide, ammonium hydroxide, NaOH, or KOH in a concentration sufficient for disrupting the binding of the nucleic acid with the compound on the solid support. In some embodiments, the eluting agent has a basic pH. In some embodiments, the eluting agent has a pH of greater than about 9, greater than about 10, greater than about 11, about 9 to about 12, about 9.5 to about 12, about 10 to about 12, or about 9 to about 11. Preferably, the pH of the eluting agent is above 10. Exemplary eluting agents comprise 1% or greater ammonia, 15 mM or greater KOH (e.g., 25 mM KOH, 35 mM KOH, 40 mM KOH, or 50 mM KOH), or 15 mM or greater NaOH (e.g., 25 mM NaOH, 35 mM NaOH, 40 mM NaOH, or 50 mM NaOH). As described herein, the use of high pH to elute nucleic acid such as DNA is unique especially to the cartridges described herein and provides improved speed and performance of the disclosed methods. Speed is provided by the rapid neutralization of acidic ammonium ions by the high concentration of hydroxide ions. A further advantage of the high pH is the denaturing effect of KOH on captured DNA or RNA. Double stranded structures and other secondary structures are disrupted, but can re-nature when neutralized for example, with Tris HCl. The cartridges described herein allows for rapid neutralization of eluted DNA or RNA in KOH/NaOH. A separate Tris reagent (such as in the form of a bead) can be provided to react with the KOH/NaOH eluent instantly to produce a final pH of about 8.5 for downstream PCR or other nucleic acid assays. In some embodiments, the eluting agent has a pH of less than about 9, less than about 8.5, or less than about 8.

In some embodiments, the eluting agent comprises a polyanion. The polyanion is generally a polymer comprising a plurality of anionic groups. In some embodiments, the anionic groups are phosphate, phosphonate, sulfate, or sulfonate groups, or combinations thereof. In some embodiments, the polyanion is a polymer negatively charged at pH above about 7. Both synthetic polyanions and naturally occurring polyanions can be used in the methods disclosed herein. In some embodiments, the polyanion is carrageenan. In other embodiments, the polyanion is a carrier nucleic acid. A carrier nucleic acid, as used herein, is a nucleic acid which does not interfere with the subsequent detection of the concentrated nucleic acid, for example, by PCR. Exemplary carrier nucleic acids include poly rA, poly dA, herring sperm DNA, salmon sperm DNA, and others well known to persons of skilled in the art. In some embodiments, the eluting agent comprises carrageenan and an alkali metal hydroxide, for example, NaOH or KOH.

Overall, strands of nucleic acid including DNA and RNA are readily captured on the solid support surfaces via the affinity reagent and washed free of impurities at pH 5 or greater. For alkylamine containing affinity reagents, for example, nucleic acid can be eluted efficiently with high pH buffers (8.5-12.5) or with at least 50 mM KOH as evidenced by PCR assay described herein.

For affinity reagents having a photocleavable group, the nucleic acid can be optionally released by photochemically or thermally cleaving the adducts. In some instances, the method of isolating nucleic acid includes eluting the nucleic acid from the solid support comprising heating the concentrated (bonded) nucleic acid to a temperature of 100° C. or less, 95° C. or less, 85° C. or less, 75° C. or less, 65° C. or less, 55° C. or less; sonicating the nucleic acid; photochemically cleaving the compound; or a combination thereof, in the presence of an eluting agent.

The nucleic acids isolated using the methods and compositions described herein are of suitable quality to be amplified to detect and/or to quantify one or more target nucleic acid sequences in the sample. Indeed, the nucleic isolation methods and compositions described herein are applicable to use in basic research aimed at the discovery of gene expression profiles relevant to the diagnosis and prognosis of disease. The methods are also applicable to the diagnosis and/or prognosis of disease, the determination particular treatment regiments, and/or monitoring of treatment effectiveness.

### Detection of Nucleic Acid

The methods described herein simplify isolation of nucleic acids from biological samples and efficiently produce isolated nucleic acids well-suited for use in RT-PCR systems. In some embodiments, the nucleic acids isolated from a nucleic acid-containing sample using the methods described herein can be detected by any suitable known nucleic acid detection method. Accordingly, methods for detecting a nucleic acid in a biological sample are disclosed. The detection method can comprise nucleic acid amplification. In some embodiments, after releasing and eluting the nucleic acid from the solid support with an eluting agent, the methods for detecting a nucleic acid can include combining the eluate with PCR reagents, which may be present in a cartridge as lyophilized particles. In some embodiments, the PCR uses Taq polymerase with hot start function, such as AptaTaq (Roche, Switzerland). The polymerase chain reaction can be a nested PCR, an isothermal PCR, gradient PCR, qPCR, reverse-transcriptase PCR, real-time PCR, multiplex PCR, nucleic acid sequence-based amplification (NASBA), transcription-mediated amplification (TMA), ligase chain reaction (LCR), rolling circle amplification (RCA), or strand displacement amplification (SDA).

In certain embodiments, the method for detecting nucleic acid in a biological sample obtained from a subject can comprise placing the biological sample in a cartridge body as described herein, wherein the cartridge body comprising a plurality of chambers in fluidic communication, a reaction tube configured for amplification of the nucleic acid by thermal cycling, and a filter in the fluidic path between the plurality of chambers and the reaction tube; lysing cells with lysis reagents present within at least one of the plurality of chambers and capturing DNA released therefrom; and amplifying the DNA with primers and probes for detecting the presence of the nucleic acid.

In some embodiments, target nucleic acids, such as coronavirus such as α-coronavirus, β-coronavirus, or SARS-CoV-2, adenovirus, Chlamydia pneumoniae, Influenza A, Influenza B, metapneumovirus, rhinovirus/enterovirus, mycoplasma, Bordetella spp., parainfluenza, and respiratory syncytial virus (RSV), hantavirus, cytomegalovirus, coxsackie virus, herpes simplex virus, echovirus, influenza virus C, Streptococcus pneumoniae, Chlamydia pneumoniae, Moraxella catarrhalis, Haemophilus influenzae, Haemophilus parainfluenzae, a group A streptococcus, Streptococcus pyogenes, Klebsiella pneumoniae, a Pseudomonas species, a Neisseria species, Histoplasnia capsulatum, Coccidioides immitis, Blastomyces dermatitidis, Paracoccidioides brasiliensis, a Candida species, an Aspergillus species, a Mucor species, Cryptococcus neoformans, or Pneumocystis carinii biomarkers and/or optional controls, can be detected. The target nucleic acids can be detected by (a) contacting nucleic acid from the sample with a set of primers and optional probes for detecting the presence of the desired target nucleic acids, (b) subjecting the nucleic acid, primers, and optional probes to amplification conditions; (c) detecting the presence of any amplification product(s), optionally via real-time PCR, melt curve analysis, or a combination thereof, and (d) optionally identifying the presence of the target nucleic acid in the sample, based on detection of the amplification product(s) or lack thereof.

In some embodiments, the amplification method comprises an initial denaturation at about 90° C. to about 100° C. for about 1 to about 10 min, followed by cycling that comprises denaturation at about 90° C. to about 100° C. for about 1 to about 30 seconds, annealing at about 55° C. to about 75° C. for about 1 to about 30 seconds, and extension at about 55° C. to about 75° C. for about 5 to about 60 seconds. In some embodiments, for the first cycle following the initial denaturation, the cycle denaturation step is omitted. The particular time and temperature will depend on the particular nucleic acid sequence being amplified and can readily be determined by a person of ordinary skill in the art.

In some embodiments, the isolation and detection of a nucleic acid is performed in an automated sample handling and/or analysis platform. In some embodiments, commercially available automated analysis platforms are utilized. For example, in some embodiments, the GeneXpert system (Cepheid, Sunnyvale, Calif.) is utilized. However, the present invention is not limited to a particular detection method or analysis platform. One of skill in the art recognizes that any number of platforms and methods may be utilized. The GeneXpert system utilizes a self-contained, single use cartridge. Sample extraction, amplification, and detection of a nucleic acid can all be carried out within this self-contained “laboratory in a cartridge.”

Examples of other approaches that can be employed in the methods describe herein include bead-based flow cytometric assay. See Lu J. et al. (2005) Nature 435:834-838, which is incorporated herein by reference for this description. An example of a bead-based flow cytometric assay is the xMAP® technology of Luminex, Inc. See www.luminexcorp.com/technology/index.html. Another approach uses microfluidic devices and single-molecule detection. See U.S. Pat. Nos. 7,402,422 and 7,351,538 to Fuchs et al, U.S. Genomics, Inc., each of which is incorporated herein by reference in its entirety. Yet another approach is simple gel electrophoresis and detection with labeled probes (e.g., probes labeled with a radioactive or chemiluminescent label), such as by northern blotting.

While in some embodiments the extracted nucleic acids are used in amplification reactions, other uses are also contemplated. Thus, for example, the isolated nucleic acids or their amplification product(s) can be used in various sequencing or hybridization protocols including, but not limited to nucleic acid-based microarrays and next generation sequencing.

Readily automated approaches are of great interest. The methods described herein can be carried out in a substantially automated manner using a commercially available nucleic acid amplification system. Exemplary nonlimiting nucleic acid amplification systems that can be used to carry out the methods of the invention include the GENEXPERT® system, a GENEXPERT® Infinity system, and GENEXPERT® Xpress System (Cepheid, Sunnyvale, Calif.). In some embodiments, the amplification system may be available at the same location as the individual to be tested, such as a health care provider's office, a clinic, or a community hospital, so processing is not delayed by transporting the sample to another facility. Assays according to the method described herein can be completed in under 3 hours, in some embodiments, under 2 hours, in some embodiments, under 1 hour, in some embodiments, under 45 minutes, in some embodiments, under 35 minutes, and in some embodiments, under 30 minutes, using an automated system, for example, the GENEXPERT® system. The GENEXPERT® utilizes a self-contained, single-use cartridge. Sample extraction, amplification, and detection may all carried out within this self-contained sample cartridge as described herein.

Prior to carrying out amplification reactions on a sample, one or more sample preparation operations are performed on the sample. Typically, these sample preparation operations will include such manipulations as extraction of intracellular material, e.g., nucleic acids from whole cell samples, viruses and the like to form a crude extract, additional treatments to prepare the sample for subsequent operations, e.g., denaturation of contaminating (e.g., DNA binding) proteins, purification, filtration, desalting, and the like. Liberation of nucleic acids from the sample cells or viruses, and denaturation of DNA binding proteins may generally be performed by chemical, physical, or electrolytic lysis methods. For example, chemical methods generally employ lysing agents to disrupt the cells and extract the nucleic acids from the cells, followed by treatment of the extract with chaotropic salts such as guanidinium isothiocyanate or urea to denature any contaminating and potentially interfering proteins. Generally, where chemical extraction and/or denaturation methods are used, the appropriate reagents may be incorporated within a sample preparation chamber, a separate accessible chamber, or may be externally introduced. Preferably, sample preparation is carried out in only one step or no more than two steps. As described herein, the methods simplify isolation of nucleic acids from biological samples and efficiently produce isolated nucleic acids well-suited for use in RT-PCR systems.

The methods for detecting nucleic acid described herein can be effected without transporting the sample from the site where the sample is collected. For example, the method can be carried out at a P° C. diagnosis location. Locations for the P° C. diagnosis include a patient care setting, preferably a hospital, an urgent care center, an emergency room, a physician's office, a health clinic, or a home. In some instances, the presence or absence of a nucleic acid can be detected within the biological sample within 75 minutes or within 60 minutes of collecting the sample from the subject.

### Discrimination of Infectious and Non-Infection (or Live and Dead) Nucleic Acid

Certain aspects of the present disclosure generally relate to compositions, devices, and methods for determining viruses such as coronaviruses. For instance, some aspects are directed to using the compositions, devices, and methods disclosed herein for determining viruses. Within the compositions and devices disclosed herein (comprising a solid support with an affinity reagent, wherein the affinity reagent comprises a first moiety that interacts with the solid support and a second moiety that interacts with nucleic acid), free RNA or other nucleic acids can preferentially partition onto the solid support, while intact viruses may be present in solution (such as in a filtrate or wash buffer) or in both solution and on the solid support. Accordingly, free RNA or other nucleic acids may be preferentially removed, e.g., as compared to intact RNA or other nucleic acids present within a virus. In some cases, the solution phase containing intact viruses can be analyzed to determine the infectiousness, e.g., of a sample arising from a subject. This may be useful, for example, for distinguishing subjects who are capable of spreading an infection from those who are not infectious.

One aspect of the present disclosure is concerned with the detection of whole virus particles in biological samples containing a mixture of said whole virus particles, other virus components such as RNA (or other nucleic acids) that are recognized by a molecular detection system, e.g., RT-PCR, as belonging to the virus, and other biological or non-biological components that are not recognized by a molecular detection system as belonging to the virus. Some embodiments include techniques for distinguishing positive molecular detection of a whole virus particle and viral components using the same molecular detection system. This may provide for the determination of samples capable of further infection from those that are not, which may, e.g., be useful for clinically separating patients who are capable of spreading an infection from those who are sick but otherwise non-infectious.

Certain embodiments rely on spatial separation of whole virus particles from other components of the same virus, e.g., RNA. Particularly, free nucleic acids such as RNA (e.g., not contained within a virus) can be partitioned preferentially onto the solid support together with the affinity reagent, e.g., due to their net negative electrical charge, while whole virus particles may be partitioned into a solution phase within the compositions and devices herein, such as within the filtrate (or wash solution) of an aqueous buffer system used to bind the free nucleic acid onto the solid support. In some cases, if a sample contains both free RNA from dead viruses and whole functional virions capable of further infection, that sample could be detected by simple molecular testing of the phase in which free RNA is not substantially present.

In one set of embodiments, whole viruses may be distributed in both solution and on the solid support. However, in other embodiments, whole viruses may be selectively or preferentially partitioned, e.g., to the solution phase of the compositions and devices herein. For instance, at least 50%, at least 60%, at least 70%, at least 80%, at least 90%, at least 95%, or at least 99% of the whole viruses may be found in the solution phase of the compositions and devices herein. In addition, in some embodiments, free RNA (or other nucleic acids) is preferentially partitioned on the solid support of the compositions and devices herein. For example, at least 70%, at least 75%, at least 80%, at least 85%, at least 90%, at least 95%, at least 96%, at least 97%, at least 98%, at least 99%, or all of the free RNA (or other nucleic acids) of the free nucleic acid molecules may be present on the solid support of the compositions and devices herein.

In certain embodiments, an assay of the solution phase (and/or nucleic acid on the solid support) may be used to determine the presence of nucleic acids. Examples of such assays include, but are not limited to, RT/PCR or other detection techniques (e.g., sequencing) known to those of ordinary skill in the art. In some embodiments, aliquots of the solution phase of the compositions and devices herein may be removed for analysis, although in other embodiments, the solution phase can be determined within the device (such as a sample cartridge) itself, in situ, i.e., without necessarily removing aliquots of the solution phase and using for analysis.

Examples of viruses that can be determined using the compositions, devices, and methods disclosed herein include, but are not limited to, coronaviruses, influenza viruses, or other viruses such as those described herein. In one set of embodiments, a sample cartridge, such as those described herein, is used. A sample, e.g., of a biological fluid taken from a subject, may be analyzed to determine whether a species of virus is present (e.g., SARS, MERS, COVID-19, etc.), and/or a type of virus is present (e.g., a coronavirus). The biological fluid may also be collected from a subject. Biological fluids may, in some cases, be processed for further use. Specific viruses, in certain embodiments, can be determined in the biological fluid. In addition, in some cases, different types of viruses may be distinguished from each other (e.g., a coronavirus versus an influenza virus).

Further non-limiting examples of viruses include infectious viruses, such coronaviruses or influenza viruses. Other non-limiting examples include adenoviruses, coxsackieviruses, Epstein-Barr viruses, hepatitis viruses (A, B, and C), herpes simplex viruses (types 1 and 2), cytomegaloviruses, herpes viruses (type 8), HIV, measles viruses, mumps viruses, papilloma viruses, parainfluenza viruses, polioviruses, rabies viruses, respiratory syncytial viruses, rubella viruses, varicella-zoster viruses, etc.

In some embodiments, a coronavirus may be determined. Examples of coronaviruses include, but are not limited to, HCoV-229E, HCoV-0C43, SARS-CoV, HCoV-NL63, HKU1, MERS-CoV, or SARS-CoV-2. In some cases, one or more proteins of the coronavirus may be used to determine the virus, e.g., by interaction with a binding moiety that is able to bind to the proteins, or a targeting species. For example, certain immunochemical assays can be used in some cases, e.g., ELISA. Other assay methods may be used to determine species within the solution phase or solid support of the device will often depend upon the identity and type of viruses or other viruses present. Examples of other suitable assay techniques include, but are not limited to, spectroscopic, chemical, fluorescent, radiological and enzymatic assays. In some cases, a virus may be determined by determining a peptide or protein associated with the virus. Non-limiting examples, include peplomers, envelope proteins, membrane proteins, nucleocapsids, spike proteins, or hemagglutinin or neuraminidase. Such peptides or proteins may be determined, for example, using suitable antibodies able to interact with these. For example, certain immunochemical assays can be used in some cases, e.g., ELISA. In addition, other peptide or protein detection techniques can be used in certain embodiments. These include, but are not limited to, direct spectrophotometry (e.g., monitoring the absorbance at 280 nanometers) and dye binding reactions with Coomassie Blue G-250 or fluorescamine, o-phthaldialdehyde, or other dyes and/or reagents. Free nucleic acids may also be determined using sequencing and other techniques such as those described herein.

In some embodiments, an influenza virus may be determined. Influenza viruses include genera such as Influenza virus A, Influenza virus B, Influenza virus C, Influenza virus D, Isavirus, Thogotovirus, and Quaranjavirus. Examples of influenza A viruses include, but are not limited to, H1N1, H1N2, H2N2, H3N1, H3N2, H3N8, H5N1, H5N2, H5N3, H5N8, H5N9, H7N1, H7N2, H7N3, H7N4, H7N7, H7N9, H9N2, H1ON7, etc.

Examples of Influenza B viruses include, but are not limited to, Victoria and Yamagata. In some cases, one or more proteins of the influenza virus may be used to determine the virus, e.g., by interaction with a binding moiety or a targeting species. Non-limiting examples of such virial proteins include hemagglutinin, neuraminidase, membrane proteins, glycoproteins, nucleocapsids, and such the like. In addition, in some cases, the nuclear material of the virus (for example, RNA) may be determined, e.g., by interaction with a virus-binding moiety or a targeting species.

The sample of biological fluid may comprise fluids as disclosed herein, such as whole blood, blood serum, blood plasma, saliva, nasal fluid, sputum, urine, CNS fluid, breast nipple aspirate fluid, cerebral spinal fluid, semen, or the like. In some cases, the subject is one that is suspected of being infected with a virus. For example, the subject may have previously been exposed to someone having the virus, or may at least be suspected of potentially having the virus. In addition, in certain embodiments, the subject may not be suspected of potentially having a virus (e.g., the fluid may be collected during routine screening).

In one aspect, a sample cartridge such as those discussed herein may be used to partition free nucleic acids, e.g., arising from dead or damaged virion particles, that may be present in a sample. For instance, a sample cartridge may be used to separate or concentrate free nucleic acids such as DNA or RNA, e.g., onto the solid support of the sample cartridge. In addition, in some cases, the amount or concentration of free nucleic acids in a sample may be determined, and optionally compared to RNA (or other nucleic acids) present within intact viruses. The type, amount, and/or concentration of RNA (or other nucleic acids) within one or more phases (solid support or solution phase) may be determined using any suitable technique, such as via polymerase chain reaction (PCR), sequencing and/or other assay techniques including those described herein. The techniques for determining intact viruses and free nucleic acids may be the same or different. As a non-limiting example, as discussed herein, intact viruses may partition in the solution phase, while RNA (or other nucleic acids) that are not present within intact viruses may preferentially partition to the solid support.

In certain embodiments, techniques for determining intact viruses and free nucleic acids such as those described herein may be related to the severity of the disease, the stage of illness, the infectiousness of the subject, or the like. As a non-limiting example, a sample may be partitioned between the solid support and solution phase of a sample cartridge such as has been described herein. After equilibration, aliquots of the solution phase can be taken and processed in some fashion, e.g., to cause any intact viruses that might be present to release their nucleic acids into solution. However, in some cases, the virus may be determined within the sample cartridge itself, in situ, i.e., without necessarily removing aliquots of one or more of the phases from the sample cartridge to be analyzed. The nucleic acids in the solution phase can be determined, e.g., using PCR or other sequencing techniques. In some cases, the amount of a particular nucleic acid sequence (e.g., the RNA of the virus) may be determined in the solution phase, and used to determine the presence of intact viruses within the sample. This method can be particularly useful since the solid support of the sample cartridge is selectively preferential to free nucleic acids, e.g., such that at least 50%, at least 55%, at least 60%, at least 65%, at least 70%, at least 75%, at least 80%, at least 85%, at least 90%, at least 95%, at least 99%, or all of free nucleic acid is present on the solid support. In some embodiments, the differences can be quantified and used to determine the relative amount or concentration of intact virus present within the sample.

Such systems can be used, for example, to distinguish between positive cases only (e.g., as determined by the presence of RNA or other free nucleic acids) and positive infective cases (e.g., as determining using free nucleic acids plus intact viruses). This can be achieved, for example, since one phase (solution phase) may contain nucleic acids arising from intact viruses, while another phase (solid support) may also contain free RNA, or other nucleic acids (e.g., DNA). Thus, in certain embodiments, determining an intact virus within a solution phase and a nucleic acid on the solid support may advantageously indicate positivity and infectiousness.

As discussed herein, a solid support together with an affinity reagent can be used in various aspects of the disclosure to determine one or more viruses (or other species). In general, the properties of the solid support together with the affinity reagent that contribute to solute partitioning include parameters as described in greater detail herein. However, since nucleic acids such as RNA are generally (negative) charged molecules, one parameter that influences its partitioning behavior between the two phases is the difference in the ionic composition of the phases in the partitioning system, e.g., via salt as an additive and its distribution.

As mentioned herein, certain aspects of the present disclosure are generally directed to the investigation of the state of a virus, although the disclosure is not limited to only viruses. Other embodiments can be applied to essentially any molecular species and/or interaction, whether biological, biochemical, chemical, or other species, and those of ordinary skill in the art will understand how the disclosure can be used in the context of other molecules. Accordingly, it is to be understood that whenever “virus” is used in the description herein, any other biological or non-biological molecule also can be used or studied in other embodiments. For example, biological sample may comprise human, bacterial, fungal, animal, or plant nucleic acid material.

In some cases, partitioning of the virus in the sample cartridge can be surprisingly rapid. For example, in some cases, substantial partitioning may occur in less than 1 hour, less than 30 minutes, less than 15 minutes, less than 10 minutes, less than 5 minutes, less than 3 minutes, less than 2 minutes, or less than 1 minute. In some cases, within these times, sufficient partitioning may have occurred to allow for determination of the virus and/or free nucleic acid as discussed herein, e.g., in one or more phases of the sample cartridge.

The following examples are for illustration purposes only, and are not meant to be limiting in any way.

### EXAMPLES

### Example 1: CVD with Alkylsilanes

Instrument: Lindberg/Blue M Vacuum Oven (Thermo Scientific) was used in this example. The vacuum port of the vacuum oven was connected to a Hi-Vac Trap assembly (Ace glass 8775-16), a vacuum gauge (Ace glass 14034-36), and then a vacuum pump (Edwards RV12). The vacuum oven was installed inside a fumehood.

Materials: Silicon substrate in size of 1×3″ with 300 nm SiO₂ layer on top surface was used in this example. High purity chemical reagent was used from n-decyltriethoxysilane (Gelest SID2665.0), n-octyltrimethoxysilane (Gelest SIO6715.5), or hexyltriethoxysilane (Gelest SIH6167.5).

CVD process: The vacuum oven was heated to stabilize at 120° C. Then a piece of 1×3″ Si substrate was loaded onto the perforated shelf in the oven, and 0.6 ml chemical reagent was dispensed in an Al dish and loaded at the bottom of the oven chamber. The oven chamber was immediately pumped down from the atmosphere pressure. The vacuum valve (between the trap and the oven chamber) was closed when the vacuum pressure shown on the gauge was less than 15 Torr. The process was dwelled for 90 min, then the chamber pumped down to 1 Torr and followed by venting to atmosphere pressure. The treated Si slide was taken out of chamber and cooled down to room temperature. Hydrophobic surface was expected on the treated slide due to the surface modification with alkyl functional groups.

### Example 2: Co-CVD with Azidosilane and Alkylsilane

Instrument: Lindberg/Blue M Vacuum Oven (Thermo Scientific) is used in this example. The vacuum port of the vacuum oven was connected to a Hi-Vac Trap assembly (Ace glass 8775-16), a vacuum gauge (Ace glass 14034-36), and then a vacuum pump (Edwards RV12). The vacuum oven was installed inside a fumehood.

Materials: Silicon substrate in size of 1×3″ with 300 nm SiO₂ layer on top surface is used in this example. The high purity chemical reagent is used from n-decyltriethoxysilane (Gelest SID2665.0), 3-azidopropyltriethoxysilane (Gelest SIA0777.0)

CVD process: The vacuum oven was heated to stabilize at 120° C., then a piece of 1×3″ Si substrate was loaded onto the perforated shelf in the oven, and 0.6 ml of 3-azidopropyltriethoxysilane was dispensed in an Al dish and loaded at the bottom of the oven chamber. The oven chamber was immediately pumped down from the atmosphere pressure. The vacuum valve (between the trap and the oven chamber) was closed when the vacuum pressure shown on the gauge was less than 2.7 Torr. The process was dwelled for 90 min, then the chamber pumped down to 1 Torr and vented to atmosphere pressure. Secondly, another fresh 0.6 ml of 3-azidopropyltricthoxysilane was dispensed in an Al dish and loaded at the bottom of the oven chamber. The oven chamber was immediately pumped down from the atmosphere pressure. The vacuum valve (between the trap and the oven chamber) was closed when the vacuum pressure shown on the gauge is less than 2.7 Torr. The process was dwelled for 90 min, then the chamber pumped down to 1 Torr and vented to atmosphere pressure. Lastly, 0.6 ml n-Decyltriethoxysilane was dispensed in an Al dish and loaded at the bottom of the oven chamber. The oven chamber was immediately pumped down from the atmosphere pressure. The vacuum valve (between the trap and the oven chamber) was closed when the vacuum pressure shown on the gauge was less than 2.7 Torr. The process was dwelled for 90 min, then the chamber pumped down to 1 Torr and vented to atmosphere pressure. The treated Si slide was taken out of chamber and cooled down to the room temperature. The treated surface was modified with both azido- and alkyl-functional groups.

### Example 3: Serial CVD with Azidosilane and Alkylsilane

Instrument: Lindberg/Blue M Vacuum Oven (Thermo Scientific) was used in this example. The vacuum port of the vacuum oven was connected to a Hi-Vac Trap assembly (Ace glass 8775-16), a vacuum gauge (Ace glass 14034-36), and then a vacuum pump (Edwards RV12). The vacuum oven was installed inside a fumehood.

Materials: Silicon substrate in size of 1×3″ with 300 nm SiO₂ layer on top surface was used in this example. The high purity chemical reagent was used from n-decyltriethoxysilane (Gelest SID2665.0), 3-azidopropyltriethoxysilane (Gelest SIA0777.0).

CVD process: The vacuum oven was heated to stabilize at 120° C. Then a piece of 1×3″ Si substrate was loaded onto the perforated shelf in the oven, and 0.6 ml of a mixed 3-azidopropyltriethoxysilane and n-decyltriethoxysilane in 1:1 ratio was dispensed in an Al dish and loaded at the bottom of the oven chamber. The oven chamber was immediately pumped down from the atmosphere pressure. The vacuum valve (between the trap and the oven chamber) was closed when the vacuum pressure shown on the gauge was less than 2.7 Torr. The process was dwelled for 90 min, then the chamber pumped down to 1 Torr and vented to atmosphere pressure. Secondly, another freshly mixed 0.6 ml of 3-azidopropyltriethoxysilane and n-decyltriethoxysilane in 1:1 ratio was dispensed in an Al dish and loaded at the bottom of the oven chamber. The oven chamber was immediately pumped down from the atmosphere pressure. The vacuum valve (between the trap and the oven chamber) was closed when the vacuum pressure shown on the gauge was less than 2.7 Torr. The process was dwelled for 90 min, then the chamber pumped down to 1 Torr and vented to atmosphere pressure. The treated Si slide was taken out of chamber and cooled down to the room temperature. The treated surface was modified with both azido- and alkyl-functional groups.

### Example 4: Measurement of Amine Loading

The assay was performed substantially as described in Noel et al., Bioconjugate Chemistry, 2007. Orange II dye is adsorbed/desorbed onto amine-functionalized surfaces at pH 3, and eluted at pH 10. Dye bound by filters was measured by obtaining the absorbance at 484 nm on a plate reader (Molecular Devices iD3). The amount of bound dye is proportional to the number of amine groups immobilized on the tested surface.

### Example 5: Retention of Target Analytes Using Hydrophobic Support and Soluble Affinity Reagent

C8 filter (SPE C8-47 mm, Restek) was punched out into smaller disks at 5/16″ diameter and placed inside spin columns (EconoSpin, Epoch Life Science). A water solution of butylamine (Sigma Aldrich) was prepared at 1.5 mM and 15 mM concentrations. 500 μL of this solution was loaded on the spin column containing C8 filters, and the spin column was spun in a microfuge at 1000 ref for 5 min. Finally, the assay described in Example 4 was used to measure Orange II binding.

As shown below, negatively charged Orange II dye (used here as a proxy for DNA) is successfully captured and eluted from a hydrophobic C₈ alkyl substrate using butylamine as an affinity reagent.

| | Abs at | Dye loading |
|---|---|---|
| Sample | 484 nm | (mg/ml) |
| 10-fold diluted eluate from C8 | 0.272 | 0.0029 |
| with 1.5 mM Butylamine | | |
| 10-fold diluted eluate from C8 | 0.289 | 0.0027 |
| with 15 mM Butylamine | | |

### Example 6: Retention of DNA Analytes Using Hydrophobic Support and Soluble Affinity Reagent

Filters (Restek SPE C8 #24048 and in-house C8-functionalized Whatman GF/F, Cytiva #1825-915) were cut to size and installed into empty spin column housings. GF008 filters were plasma cleaned prior to functionalization, while GF007 filters were not. Filters were then treated with 500 uL of either pure water or 100% methanol by centrifuging the solution through the filter (2000× g for 1 minute for this and all subsequent spins) and discarding the flow-through. This was then followed by three 500 uL washes with pure water. Filters were then treated with 500 uL of a 15 mM butylamine pH 6.0 solution. 500 uL of lambda DNA (Thermo SD0011, at 30 ng/uL in 15 mM butylamine pH 6.0) was then applied and centrifuged through the filter, collecting flow-throughs for analysis. Filters were then washed three times with 500 uL of either pure water or 15 mM butylamine pH 6.0, and bound DNA was eluted by treatment with 500 uL water set to pH 12.0 with NaOH. Elutions were collected for analysis. All flow-throughs and elutions were assayed for DNA content using the Qubit 1x Broad Range DNA Kit (Thermo Q33266) following kit instructions, with 10 uL of each sample added to a final analysis volume of 200 uL.

The table below shows a summary of DNA binding and elution as measured by fluorometry. All values reported in ng/uL. SPE=Restek C8 filters, GF007=C8 functionalized GF/F, GF008=plasma cleaned, C8 functionalized GF/F. Asterisk (*) indicates filter damage/breakage during assay.

| | | | DNA in | |
|---|---|---|---|---|
| | | | flow- | DNA in |
| | | | through | eluate |
| Conditioning | Washing | Filter | (ng/uL) | (ng/uL) |
| Water | Water | SPE | 28.4 | 0.579 |
| Water | Water | GF007 | <0.2 | 2.48 |
| Water | Water | GF008 | <0.2 | 8.29 |
| Methanol | Water | SPE | 8.43 | 0.627 |
| Methanol | Water | GF007 | <0.2 | 3.72 |
| Methanol | Water | GF008 | <0.2 | 6.96 |
| Water | Butylamine | SPE | 25.5 | 1.55 |
| Water | Butylamine | GF007 | 3.17 | 9.13 |
| Water | Butylamine | GF008 | <0.2 | 18.2 |
| Methanol | Butylamine | SPE | 26.7* | <0.2 |
| Methanol | Butylamine | GF007 | <0.2 | 13.3 |
| Methanol | Butylamine | GF008 | <0.2 | 18.2 |

### Example 7: Modified Glass Fiber Filter for Removal of Background Nucleic Acid for Selective Detection of Active Viral Infection

Glass fiber filters (or other surfaces) can be modified with amino silanes which have been shown to have affinity for nucleic acid. Passing a swab sample through a glass filter (such as in a cartridge pre-filter) with such amine modification can remove any free nucleic acid from the sample. Viral particles will not have affinity to the filter, and will pass through unaffected. The test will thus only detect viral particles, and not residual RNA/DNA.

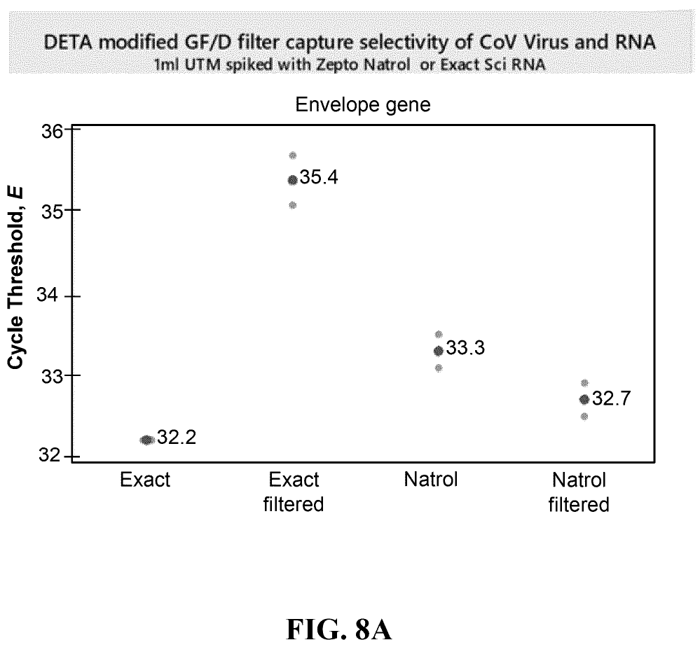

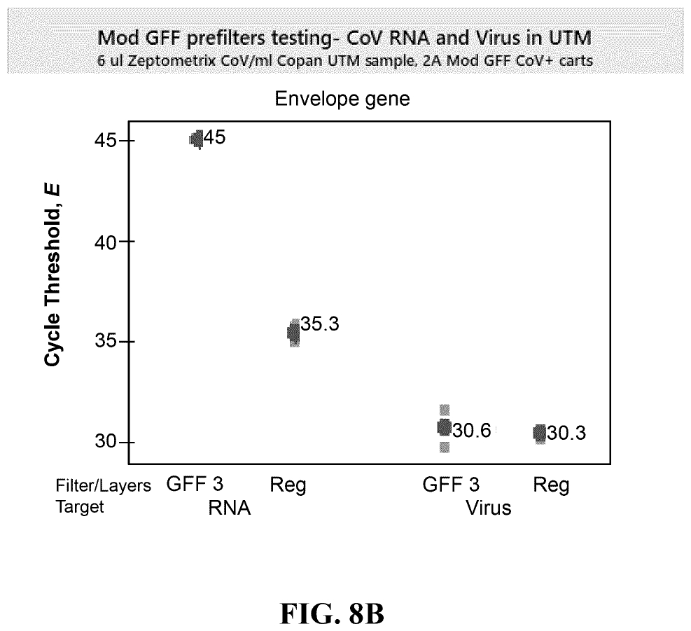

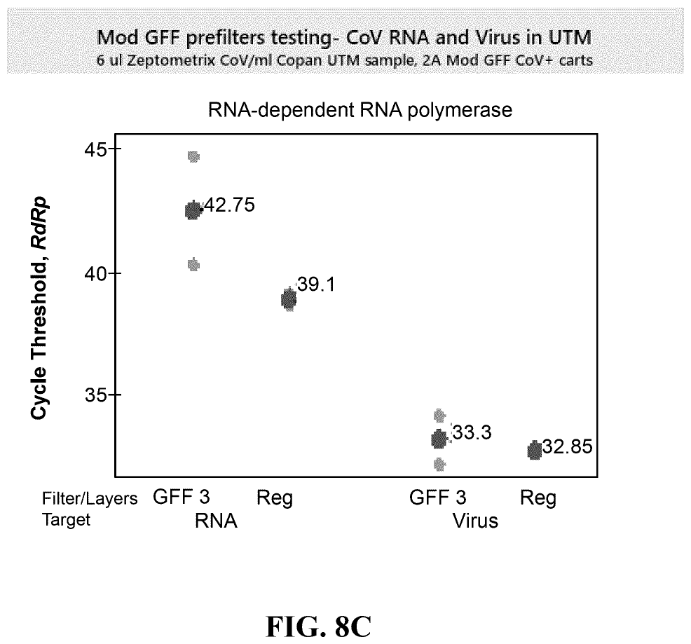

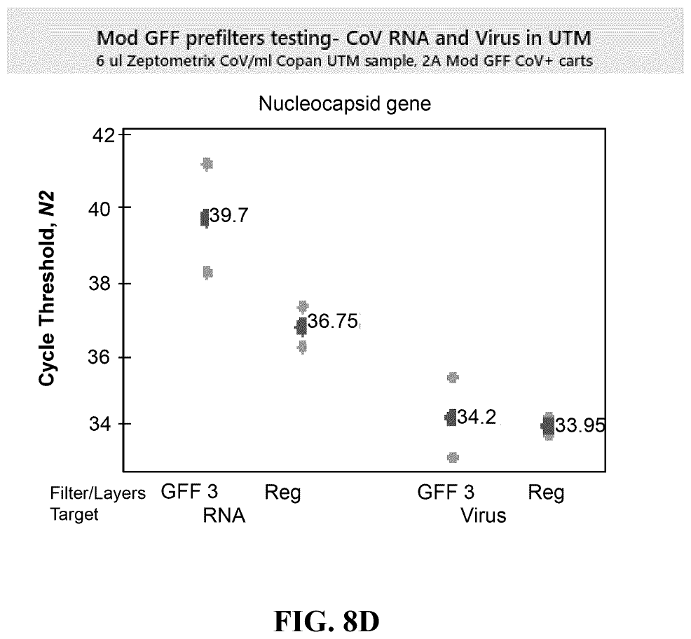

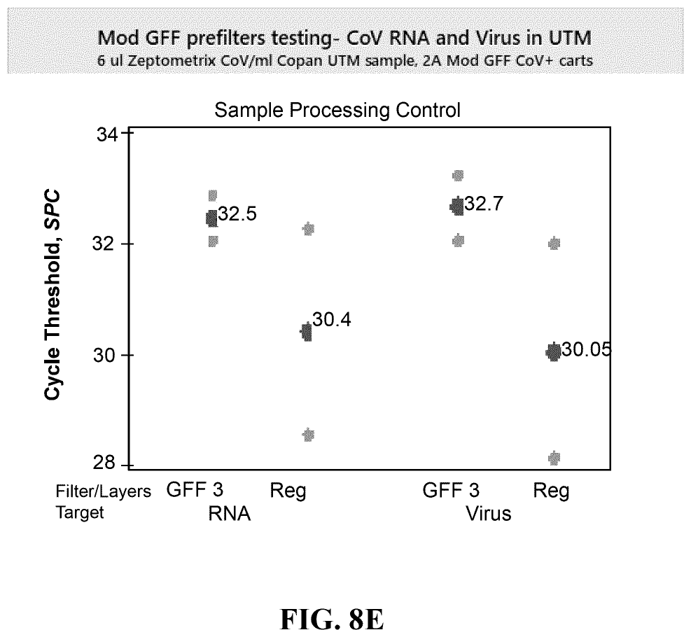

FIGS. 8A through 8E show a glass fiber filter (GF/D from Cytiva; 5 micron pore size) modified with diethylentriamino silane can capture ˜90% COVID RNA (UTM containing CoV2 RNA from Exact Sciences) while not capturing inactivated virus (Zepto Natrol from Zeptometrix).

Glass fiber filter (GF/D from Cytiva; 5 micron pore size) modified with aminopropyltriethoxylsilane (APTES) were cut into discs for assembly into prefilters for Cepheid GX cartridges. FIGS. 8B through 8E demonstrate that cartridges with 3 layers of GFF material prefilter can remove viral RNA from sample (Copan UTM media) while not impacting intact viral particles.

### Example 8: Retention of Target Analytes Using Hydrophobic Support and Branched Soluble Affinity Reagent

C8 filter (SPE C8-47 mm, Restek) was punched out into smaller disks at 5/16″ diameter and placed inside spin columns (EconoSpin, Epoch Life Science). A methanol solution of 3-dibutylamino propylamine (Sigma Aldrich) was prepared at 1.5 mM and 15 mM concentrations. 500 μL of this solution was loaded on the spin column containing C8 filters, and the spin column was spun in a microfuge at 1000 rcf for 5 min. Finally, the assay described in Example 4 was used to measure Orange II binding.

As shown below, negatively charged Orange II dye (used here as a proxy for DNA) is successfully captured and eluted from a hydrophobic C8 alkyl substrate using butylamine as an affinity reagent.

| | Abs at | Dye loading |
|---|---|---|
| Sample | 484 nm | (mg/ml) |
| 10-fold diluted eluate from C8 | 0.272 | 0.0029 |
| with 1.5 mM Butylamine | | |
| 10-fold diluted eluate from C8 | 0.289 | 0.0027 |
| with 15 mM Butylamine | | |

### Example 9: Synthesis of Linkers for Synthesizing Branched Soluble Affinity Reagent

N,N-dihexyl-3-aminopropylamine: To a suspension containing 1 g of dihexylamine (2 mmole), in 10 ml methanol and 10 ml DCM was added 10 ml of acrylonitrile. The reaction was continued for 24 h at room temperature. The solvents were removed under vacuum and the product was purified by silica gel using Ethyl acetate and Hexane as solvents. The product (0.8 g) was then dissolved in 10 ml ether and 1 g of LiAlH₄ was added. The reduction was allowed for overnight at room temperature. The reaction was stopped with dilute NaOH in water at 0° C. The reaction mixture was filtered and extracted with Ethyl acetate. The organic solvent was dried over sodium sulfate and evaporated to give 0.2 g white powder. Mass detected in ESI-MS (+ve): 243 m/z. Overall Yield: 15%.

N,N-dihexyl-3-imidazole-2-oxoamine: To a solution of imidazole propionic acid (50 mg, 0.4 mmol) in DCM (10 mL), HATU (0.89 mmol) and DIPEA (1 mmol) were added. To this dihexylamine (0.48 mmol) was added and the mixture was stirred at room temperature for 1 day. The reaction was quenched with H₂O and extracted with DCM. The organic phase was washed with H₂O and brine, dried with Na₂SO₄, and evaporated under reduced pressure to give an oil. The oil was purified by alumina column chromatography using DCM:Methanol as solvents.

### Example 10: Measurement of Amine Loading in Polar Solvents

Affinity reagent comprising 3-dibutylaminopropylamine is solubilized in water using a polar solvent such as methanol or DCM. Once solubilized the affinity reagent is applied to the columns. Orange II dye is adsorbed/desorbed onto amine-functionalized surfaces at pH 3, and eluted at pH 12. Dye bound by filters was measured by obtaining the absorbance at 484 nm on a plate reader (Molecular Devices iD3). The amount of bound dye is proportional to the number of amine groups immobilized on the tested surface.

Samples having 20% and 10% w/v affinity reagent with 3-dibutylaminopropylamine in 20% v/v methanol show similar binding to that of neat 3-dibutylaminopropylamine and 3-dibutylaminopropylamine in water. 10% (w/v) affinity reagent with 3-dibutylaminopropylamine is solubilized by 20% methanol.

### Example 11: Measurement of DNA Binding with 3-Dibutylaminopropylamine

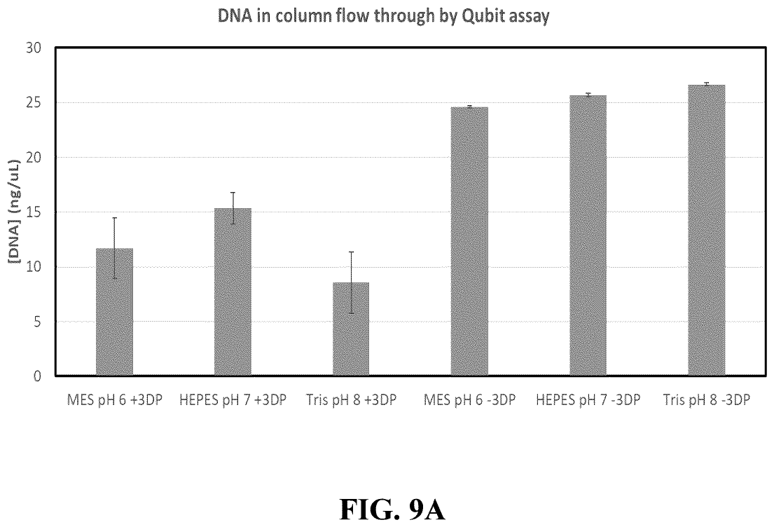

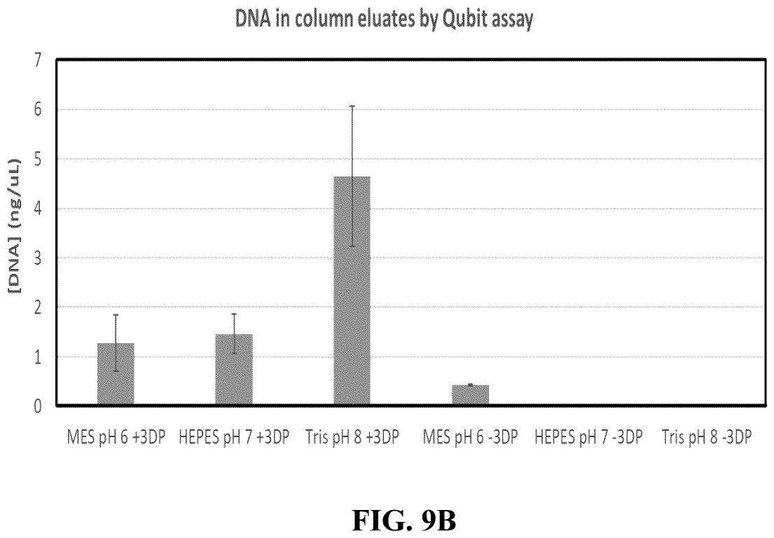

Affinity reagent comprising about 10% w/v 3-dibutylaminopropylamine in 20% v/v methanol are applied to C8 columns. The column is washed with buffer at pH 6, 7, or 8. DNA in buffer is adsorbed/desorbed onto amine-functionalized surfaces, washed three times with buffer, and eluted at pH 12. The flow-through and eluate of DNA binding was measured with Quibit assay. FIGS. 9A and 9B show plots of DNA binding and elution as measured by fluorometry. As shown, tris pH 8 provided best capture and release, reaching about 71% of load and about 22% of capture.

### Example 12: Effect of Salt on DNA Binding with 3-Dibutylaminopropylamine

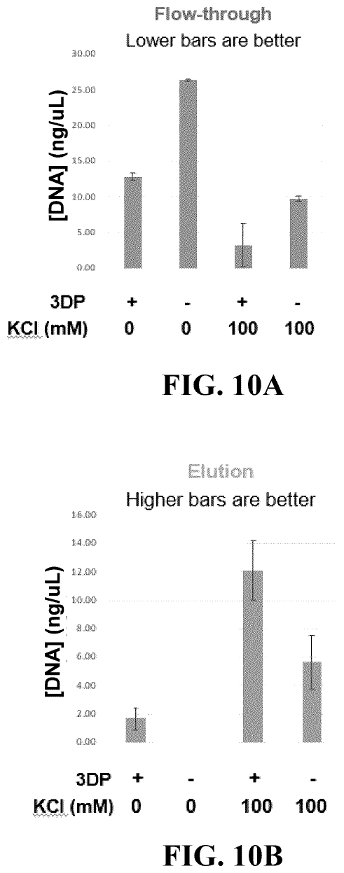

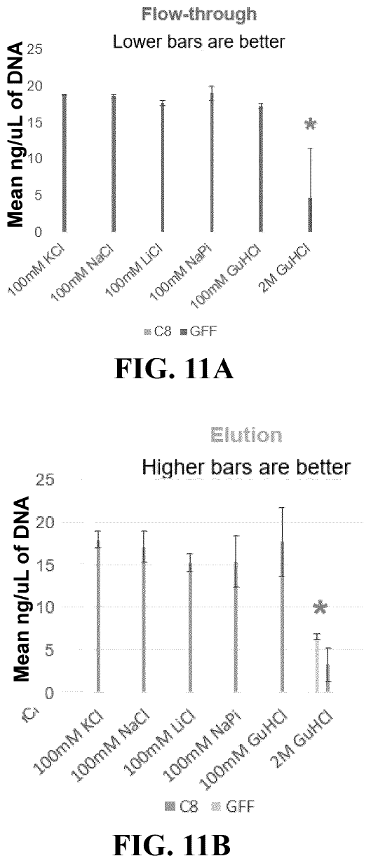

Affinity reagent comprising about 10% w/v 3-dibutylaminopropylamine in 20% v/v methanol are applied to C₈ columns. The column is washed with buffer at pH 8 and 100 mM salt (e.g., KCl, NaCl, LiCl, NaPi, and GuHCl). DNA in buffer is adsorbed/desorbed onto amine-functionalized surfaces, washed three times with buffer, and eluted at pH 12. The flow-through and eluate of DNA binding was measured with Quibit assay. FIGS. 10A and 10B demonstrate effect of salt on DNA binding and elution as measured by fluorometry. As shown, addition of KCl and similar salts improves DNA capture and recovery. FIGS. 11A and 11B demonstrate the effect of DNA binding and elution when washed with KCl NaCl, LiCl, NaPi, and GuHCl. Binding is near 100% (30 ng/uL in 500 μL lambda DNA) for C8 with any 100 mM salt. Elution is about 50% of load with various salts at a concentration of 100 mM.

### Example 13: Effect of DNA Binding with 3-Dihexyl-3-Aminopropylamine (DAP)

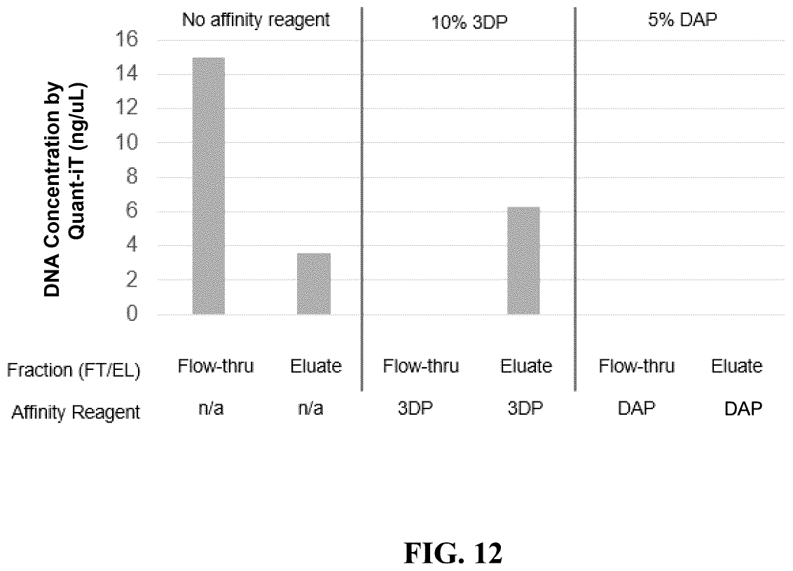

Affinity reagent comprising about 10% w/v 3-dibutylaminopropylamine or 5% w/v 3-dihexyl-3-aminopropylamine (DAP) in 20% v/v methanol are applied to filters. The filter is washed with buffer at pH 8 and 100 mM salt (e.g., KCl, NaCl, LiCl, NaPi, and GuHCl). DNA in buffer is adsorbed/desorbed onto amine-functionalized surfaces, washed three times with buffer, and eluted at pH 12. The flow-through and eluate of DNA binding was measured with Quant-iT assay. FIG. 12 compares DNA binding and elution with affinity reagent 3DP and DAP.

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that changes can be made without departing from the spirit and scope of the invention(s).

1. A method of isolating nucleic acid from a sample, the method comprising:
contacting a solid support with an affinity reagent and the sample, wherein the affinity reagent comprises:
a first moiety that interacts with the solid support,
a second moiety that interacts with nucleic acid in the sample, and
concentrating the nucleic acid onto the solid support.

2. The method of claim 1, wherein the affinity reagent further comprises a linker that interacts with a solvent and increases solubility of the affinity reagent.

3. The method of claim 1, wherein the solid support comprises a functional surface group that interacts with the first moiety of the affinity reagent, wherein the functional surface group comprises a hydrophobic binding group, a negatively charged binding group, a positively charged binding group, a polar binding group, or a combination thereof, and
optionally, wherein the functional surface group comprises a hydrophobic binding group selected from an alkyl group, a cycloalkyl group, a haloalkyl group, an aryl group, or a combination thereof.

4. The method of claim 1, wherein the functional surface group comprises an azido group and the first moiety of the affinity reagent comprises a cycloalkyne, and wherein the azido group and the cycloalkyne react to form a nitrogen-containing heterocycle.

5. The method of claim 1, wherein the functional surface group is bound to the solid support via a triazole group, a triazinyl group, an imidazole, an indole, a silane group, a silatrane group, a siloxane group, a cyclic siloxane group, a silsesquioxane group, a silazane group, or a combination thereof.

6. The method of claim 1, wherein the first moiety of the affinity reagent that interacts with the solid support comprises a hydrophobic group, a negatively charged binding group, a positively charged binding group, a polar group, or a combination thereof.

7. The method of claim 6, wherein the first moiety of the affinity reagent comprises a hydrophobic group, wherein the hydrophobic group is selected from an alkyl group, a cycloalkyl group, a haloalkyl group, an aryl group, or a combination thereof.

8. The method of claim 1, wherein the second moiety of the affinity reagent that interacts with the nucleic acid comprises an amine, a nitrogen containing heterocycle, an intercalating agent, a DNA groove binder, a peptide, an amino acid, a protein, or a combination thereof.

9. The method of claim 8, wherein the second moiety of the affinity reagent comprises spermine, methylamine, ethylamine, propylamine, ethylenediamine, diethylene triamine, 1,3-dimethyldipropylenediamine, 3-(2-aminoethyl)aminopropyl, (2-aminoethyl)trimethylammonium hydrochloride, tris(2-aminoethyl)amine, 3-dibutylamino propylamine, or a combination thereof.

10. The method of claim 2, wherein the linker of the affinity reagent further comprises one or more functional groups that interact with the solvent in the sample to increase solubility of the affinity reagent.

11. The method of claim 10, wherein the linker comprises polyethylene glycol linkers, ester-based linkers (such as branched ester-based linkers), amide-based linkers (such as branched amide-based linkers including N,N-dihexyl-3-imidazole-2-oxoamine linkers), amine-based linkers (such as 3-dibutylaminopropylamine linkers, N,N-dihexyl-3-aminopropylamine linkers), organophosphorous-based linkers (such as organophosphine-based linkers, organophosphine oxide-based linkers, organophosphinate-based linkers, organophosphoramidate-based linkers, organophosphate-based linkers, organophosphonamidate-based linkers, or organophosphonate-based linkers), glucuronic acid-based linkers, disulfide linkers, cathepsin B linkers, or combinations thereof.

12. The method of claim 1, further comprising releasing the nucleic acid concentrated on the solid support.

13. The method of claim 12, wherein releasing the nucleic acid concentrated on the solid support comprises exposing at least one of the solid support, affinity reagent, or the nucleic acid to an eluting agent, heat, sonication, conditions for photochemical cleavage, or a combination thereof.

14. The method of claim 13, wherein releasing the nucleic acid concentrated on the solid support comprises exposing at least one of the solid support, affinity reagent, or the nucleic acid to an eluting agent, optionally wherein the eluting agent has a pH greater than about 7, greater than about 8, greater than about 9, and/or a salt concentration higher than the sample.

15. A method for detecting a nucleic acid in a biological sample, comprising:
(a) isolating the nucleic acid from a sample using a method as defined in claim 1;
(b) releasing the nucleic acid from the solid support with an eluting agent; and
(c) detecting the nucleic acid.

16. A sample cartridge for isolation and detection of nucleic acid from a biological sample, the sample cartridge comprising:
a cartridge body having a plurality of chambers therein, wherein the plurality of chambers includes a sample chamber configured to receive the biological sample;
a reaction vessel fluidically coupled to the plurality of chambers and configured for amplification of nucleic acid and detection of a plurality of amplification products;
a filter disposed in the fluidic path between the plurality of chambers and the reaction vessel, wherein the filter comprises a solid support having a surface capable of binding an affinity reagent,
wherein the affinity reagent is disposed in one of the plurality of chambers and comprises a first moiety that interacts with the solid support and a second moiety that interacts with a nucleic acid from the biological sample; and
primers and/or probes disposed in one or more chambers of the plurality of chambers or reaction vessel for detection of the nucleic acid.

17. The sample cartridge of claim 16, wherein the solid support comprises a functional surface group that interacts with the first moiety of the affinity reagent,
optionally, wherein the functional surface group interacts with the first moiety of the affinity reagent covalently or non-covalently, or a combination thereof.

18. The sample cartridge of claim 16, wherein
the first moiety of the affinity reagent that interacts with the solid support comprises a hydrophobic group, a negatively charged binding group, a positively charged binding group, a polar group, or a combination thereof, and
the second moiety of the affinity reagent that interacts with the nucleic acid comprises an amine, an intercalating agent, a DNA groove binder, a peptide, an amino acid, a protein, or a combination thereof.

19. The sample cartridge of claim 16, wherein the affinity reagent further comprises a linker that interacts with a solvent and increases solubility of the affinity reagent.

20. The sample cartridge of claim 19, wherein the linker comprises a hydrophilic group selected from polyethylene glycol linkers, ester-based linkers, amide-based linkers, amine-based linkers, organophosphorous-based linkers, glucuronic acid-based linkers, disulfide linkers, cathepsin B linkers, or combinations thereof.
